(19) United States
(12) Patent Application Publication
(10) Pub. No.: US 2008/0311098 A1
(43) Pub. Date: Dec. 18, 2008

(54) COMPOUNDS AND METHODS FOR MODULATING THE IMMUNE RESPONSE AGAINST ANTIGENS

(76) Inventors: Rejean Lapointe, Laval (CA); Stephanie Lepage, Delson (CA)

Correspondence Address: GOUDREAU GAGE DUBUC, 2000 MCGILL COLLEGE, SUITE 2200, MONTREAL, QC, H3A 3H3, CA

(21) Appl. No.: 12/028,472

(22) Filed: Feb. 8, 2008

Related U.S. Application Data
(60) Provisional application No. 60/901,092, filed on Feb. 14, 2007.
(60) Provisional application No. 60/989,633, filed on Nov. 21, 2007.

(51) Int. Cl. A61K 35/12; C07H 21/00; C07K 2/00; C12N 15/63; A61P 37/04; C12N 5/00; A61K 31/7052; A61K 38/02

(52) U.S. Cl. 424/93.71; 536/23.1; 530/300; 435/320.1; 435/325; 514/44; 514/2; 435/455; 435/375

(57) ABSTRACT

Chimeric nucleic acids and polypeptides comprising an antigen or an epitope thereof are described, as well as compositions and methods to increase the presentation of an antigen or epitope by MHC class II molecules and to modulate the immune response.

### CROSS REFERENCE TO RELATED APPLICATIONS

This application claims the benefit, under 35 USC § 119(e), of U.S. Provisional Patent Application Ser. No. 60/901,092 filed on Feb. 14, 2007, and U.S. Provisional Patent Application Ser. No. 60/989,633 filed on Nov. 21, 2007, the contents of which are incorporated herein by reference in their entirety.

### FIELD OF THE INVENTION

The present invention relates to compounds and methods for modulating the immune response against antigens. More specifically, the present invention is concerned with increasing MHC class II presentation of antigenic epitopes.

### BACKGROUND OF THE INVENTION

Gp100 (also knows as Pmel 17), a melanoma/melanocyte-shared antigen, can be presented by both Major Histocompatibility Complex (MHC) class I and class II molecules when expressed endogenously by melanoma and non-melanoma cells. This implies that gp100 can reach endosomal/MHC class II compartments (MIIC) for antigen processing and presentation by MHC class II molecules. Normally, CD4⁺ T cells recognize exogenous proteins, which are ingested by antigen-presenting cells (APCs) and get degraded into peptides which can be coupled with MHC class II molecules in MIIC, which are lysosome-related organelles (Brocker et al. 1984. J. Invest. Dermatol. 82:244-247). These peptide/MHC class II complexes then migrate to the cell surface. Interestingly, an endogenous protein can sometimes reach endosomal/MIIC to be processed similarly to an exogenous protein for MHC class II-mediated presentation. However, endosomal/MIIC internal trafficking leading to MHC class II presentation remains poorly understood.

Cancer immunotherapy strategies targeting tumor antigens (TA) were mainly developed by eliciting CD8⁺ cytotoxic T lymphocytes (CTLs). Over the past decade, growing evidence has emerged from animal studies (Hung et al., 1998. J. Exp. Med.; 188:2357-2368; Surman et al., 2000. J. Immunol. 164:562-565; Corthay et al., 2005. Immunity 22:371-383.) and clinical trials (Phan et al., 2003. J. Immunother. 26:349-356; Wong et al., 2004. Clin. Cancer Res. 10:5004-5013), indicating that CD4⁺ helper T lymphocytes play an important role in initiating and maintaining immune responses against cancer (Toes et al., 1999. J. Exp. Med. 189:753-756; Wang et al., 2001. Trends Immunol. 22:269-276) by expanding effective and memory CD8⁺ T cells (Janssen et al., 2003. Nature 421:852-856; Shedlock D J and Shen H., 2003. Science 300:337-339.). Thus, optimal anti-tumor immunity might require the participation of both CD4⁺ and CD8⁺ T lymphocytes to generate a strong and durable response against cancer cells (Velders et al., 2003. Int. Rev. Immunol. 22:113-140, Gerloni M. and Zanetti M., 2005. Springer Semin. Immunopathol. 27:37-48). Similarly, optimal anti-viral immunity would require participation of both CD4⁺ and CD8⁺ T lymphocytes to generate a strong and durable response against viruses.

Considering that about 20-25% of melanomas naturally express MHC class II molecules during the process of malignant transformation (Lopez-Nevot et al., 1988. Exp. Clin. Immunogenet. 5:203-212), and perhaps more than 50% during inflammation and metastases formation (Bernsen et al., 2003. Br. J. Cancer 88:424-431; Brocker et al., 1984. J. Invest. Dermatol. 82:244-247), it is plausible that concomitant antigenic presentation by MHC class I and class II shapes anti-tumor responses. Thus, activation of tumor-specific CD8⁺ and CD4⁺ T cells may occur at the tumor site. This illustrates the importance of better defining MHC class II antigenic presentation from endogenously-expressed proteins.

Given the role played by CD4⁺ T lymphocytes in the immune responses against pathogens and tumors, there is a need to develop new strategies for enhancing CD4⁺ T cell responses. Thus, there is a need for novel compounds and methods for increasing the presentation of antigens by MHC molecules, such as MHC class II molecules.

The present description refers to a number of documents, the content of which is herein incorporated by reference in their entirety.

### SUMMARY OF THE INVENTION

In a first aspect, the present invention provides a chimeric nucleic acid comprising:

(a) a first domain comprising a nucleic acid encoding a signal peptide; and

(b)a second domain comprising a nucleic acid encoding a polypeptide comprising (i) a transmembrane domain and (ii) an antigen or an epitope thereof,

wherein at least one of the signal peptide and the transmembrane domain is that of gp100, and wherein said antigen or epitope is heterologous to at least one of signal peptide and said transmembrane domain.

In an embodiment, the above-mentioned signal peptide is a signal peptide from gp100.

In another embodiment, the above-mentioned signal peptide comprises the sequence of SEQ ID NO:3. In a further embodiment, the above-mentioned nucleic acid encoding a signal peptide comprises the sequence of SEQ ID NO:12.

In another embodiment, the above-mentioned polypeptide comprises the transmembrane domain of gp100 or of CD8.

In a further embodiment, the above-mentioned transmembrane domain comprises the sequence of SEQ ID NO:1 or 4. In a further embodiment, the above-mentioned nucleic acid encoding a transmembrane domain comprises the sequence of SEQ ID NO:10 or 13.

In another embodiment, the above-mentioned polypeptide comprises the sequence of SEQ ID NO:2. In a further embodiment, the above-mentioned polypeptide further comprises the sequence of SEQ ID NO:5. In a further embodiment, said transmembrane domain is that of CD8 and the polypeptide further comprises at the C-terminal of the transmembrane domain the sequence of SEQ ID NO:5.

In another embodiment, the above-mentioned nucleic acid encoding a polypeptide comprises the sequence of SEQ ID NO:11. In a further embodiment, the above-mentioned nucleic acid encoding a polypeptide further comprises the sequence of SEQ ID NO:14.

In another aspect, the present invention provides a polypeptide encoded by the above-mentioned chimeric nucleic acid.

In yet another aspect, the present invention provides a vector comprising the above-mentioned nucleic acid.

In another aspect, the present invention provides a cell comprising the above-mentioned nucleic acid or vector.

In another aspect, the present invention provides a composition comprising the above-mentioned chimeric nucleic acid or polypeptide, and a pharmaceutically acceptable carrier or excipient. In an embodiment, the above-mentioned composition further comprises an adjuvant.

In another aspect, the present invention provides a method for inducing or enhancing an immune response against an antigen in a subject, comprising administering to said subject a composition comprising a chimeric nucleic acid and an adjuvant, said chimeric nucleic acid comprising:

(a)a first domain comprising a nucleic acid encoding a signal peptide; and

(b)a second domain comprising a nucleic acid encoding a polypeptide comprising (i) transmembrane domain and (ii) said antigen or an epitope thereof, wherein said antigen or epitope is heterologous to at least one of said signal peptide and said transmembrane domain.

In another aspect, the present invention provides a method for enhancing MHC class-II presentation of an antigenic epitope in a cell, comprising transfecting or transforming said cell with a nucleic acid comprising:

(a)a first domain comprising a nucleic acid encoding a signal peptide; and

(b)a second domain comprising a nucleic acid encoding a polypeptide comprising (i) transmembrane domain and (ii) said antigenic epitope, wherein said antigenic epitope is heterologous to at least one of said signal peptide and said transmembrane domain.

In another aspect, the present invention provides a method for inducing or enhancing an immune response against an antigen in a subject, comprising administering to said subject cells transfected or transformed with the chimeric nucleic acid of the present invention.

In another aspect, the present invention provides a method for inducing or enhancing an immune response against an antigen in a subject, comprising administering to said subject T lymphocytes that have been co-cultured with cells transfected or transformed with the chimeric nucleic acid of the present invention.

In another aspect, the present invention provides a method of expanding T lymphocytes comprising co-culturing the lymphocytes with cells transfected or transformed with the chimeric nucleic acid of the present invention.

In another embodiment of the above-mentioned method, said cells and/or said T lymphocytes are autologous to the subject.

In an embodiment, the above-mentioned antigen or epitope thereof is derived from a tumour antigen. In a further embodiment, the above-mentioned subject has a tumour expressing the tumour antigen.

In another embodiment, the above-mentioned antigen or epitope thereof is derived from a viral antigen. In a further embodiment, the above-mentioned subject is susceptible to or has a viral infection expressing the viral antigen.

In an embodiment, the above-mentioned cell is an antigen-presenting cell (APC). In a further embodiment, the above-mentioned APC is a dendritic cell (DC).

Other objects, advantages and features of the present invention will become more apparent upon reading of the following non-restrictive description of specific embodiments thereof, given by way of example only with reference to the accompanying drawings.

This invention will be described, referring to the following specific embodiments and appended figures, the purpose of which is to illustrate the invention rather than to limit its scope.

### BRIEF DESCRIPTION OF THE DRAWINGS

In the appended drawings:

FIG. 1 shows MHC class II-restricted presentation of endogenous and exogenous gp100. A. HLA-DRβ1*0701⁺- or HLA-DRβ1*0701⁻-stimulated B lymphocytes (CD40-B) were pulsed with recombinant gp100 (gp100r), or (NY-ESO-1r), different amounts of gp100₁₇₀₋₁₉₀ peptide, i.e. a fragment specific to HLA-DRβ1*0701⁺, or a DRβ1*0701-binding control peptide (Igk₁₈₈₋₂₀₂). A gp100-specific CD⁴⁺ T cell clone was co-cultured with these target cells. B. An HLA-DRβ1*0701⁺ gp100 -deficient melanoma cell line (melFB) was retrovirally-transduced using genes encoding gp100 or green fluorescent protein (GFP). 293T cells expressing HLA-DRβ1*0701 (DR7) or DRβ1*0401 (DR4) were co-transfected with plasmids coding for gp100 or GFP and HLA-A*0201 (A2) or A*0101 (A1) to generate target cells. Gp100-specific CD4⁺ and CD8⁺ T cell clones were co-cultured with these target cells. C. Melanoma cell lines expressing or not gp100 and MHC class II molecules (HLA-DRβ1*0401 (DR4) or HLA-DRβ1*0701 (DR7)) were co-cultured with a gp100-specific CD4⁺ T cell clone. D. HLA-DRβ1*0701⁺-stimulated B lymphocytes (CD40-B) pulsed with recombinant gp100 (gp100r); HLA-DRβ1*0701+gp100-deficient melanoma cell line (MelFB) expressing gp100 or GFP; gp100-transfected 293T cells expressing HLA-DRβ1*0701 (DR7), DRβ1*0401 (DR4) or HLA-A*0201 (A2); and melanoma cell lines expressing or not gp100 or HLA-A*0201 (HLA-A2), were treated with 100 mM chloroquine (CHL) or left untreated (NT), as described in Example 1 below (Materials and Methods). Gp100-specific CD4⁺ and CD8⁺ T cell clones were co-cultured with these target cells. For A, B, C and D, supernatants were harvested after 20-hour co-culture, and IFN-y secretion was determined by ELISA. Results are representative of 5 independent experiments;

FIG. 2 shows co-localization of gp100 and LAMP-1. 293T cells transfected with plasmids coding for gp100 or tyrosinase and a melanoma cell line expressing gp100 (MelFB-gp100) were permeabilized and double-stained with an Alexa Fluor™-488-conjugated anti-gp100 antibody and an Alexa Fluor™-568-conjugated anti-LAMP-1 antibody. The cells were analyzed by laser scanning confocal microscopy. White arrows, in merged images, revealed the co-localization of gp100 and LAMP-1. Results are representative of 5 independent experiments;

FIG. 3 shows the mapping of gp100-derived targeting sequences involved in MHC class II presentation. A. Plasmids encoding gp100 mutants and HLA-A*0201 (A2) or A*0101 (A1) were co-transfected in 293T cells expressing HLA-DRβ1*0701 (DR7) or DRβ1*0401 (DR4). Gp100-specific CD4⁺ and CD8⁺ T cell clones were co-cultured with transfected cells, and peptide presentation was evaluated on the basis of IFN-γ secretion, as determined by ELISA. Data from gp100 mutants are presented as a percentage of IFN-γ secretion compared to wild-type gp100, normalized to 100% (average of 10 independent experiments). The Di-leucine Motif at the C-terminal of gp100 is presented (SEQ ID NO: 16). Polypeptide sequences N-terminal and C-terminal of the CD8 transmembrane domain in the gp100 mutant ΔYYCD8 are presented as (SEQ ID NO: 17) and (SEQ ID NO: 18) respectively. B. Data from panel A are presented as a MHC class II/ MHC class I antigen presentation efficiency ratio. C. Amino acid sequences of the transmembrane domain from CD8 (boxed and shaded) (SEQ ID NO: 4) within a larger region of CD8 (SEQ ID NO: 19); and the transmembrane domain (boxed and shaded)_(SEQ ID NO: 2), the ΔYY region of gp100 (underlined) (SEQ ID NO: 5) and the Di-leucine Motif (boxed) (SEQ ID NO: 16) are presented within a larger region of gp100 (SEQ ID NO: 20). D. Gp100 expression levels were determined by Western blot analysis with a gp100-specific antibody. Since the epitope recognized by this antibody is located at the amino-terminus of gp100, the expression level of gp100-ΔSS could not be determined. E. Gp100 cell surface expression was evaluated by flow cytometry (Cell surface expression). Total gp100 expression was also evaluated, by permeabilizing transfected cells prior to staining (Total gp100 ). Gp100 cell surface expression for all gp100 mutants is summarized in panel B. (Data are representative of 6 independent experiments);

FIG. 4 shows gp100 cell surface expression in melanoma cell lines. Gp100 cell surface expression from different melanoma cell lines was evaluated by flow cytometry (Cell surface). Total gp100 expression was also evaluated, by permeabilizing cells prior to staining (Total). Data are representative of 3 independent experiments;

FIG. 5 shows endosomal localization of gp100 mutant. A. 293T cells transfected with plasmid coding for gp100 or gp100 mutants were permeabilized and double-stained with an Alexa Fluor™-488-conjugated anti-gp100 antibody and an Alexa Fluor™-568-conjugated anti-LAMP-1 antibody. The cells were analyzed by laser scanning confocal microscopy. B. Gp100 -transfected 293T cells and a melanoma cell line expressing gp100 (MelFB-gp100 ) were permeabilized and stained with an anti-gp100 antibody conjugated with Alexa Fluor-488, an anti-HLA-DR antibody conjugated with Alexa Fluor™-568 and an anti-LAMP-1 antibody conjugated with Alexa Fluor™-647. The cells were analyzed by laser scanning confocal microscopy. The central image revealed co-localization of gp100, LAMP-1 and MHC class II molecule HLA-DR. Results are representative of 2 independent experiments;

FIG. 6 shows endosomal mobilization of GFP, and presentation of minimal MHC class II and class I epitopes, using gp100-targeting sequences. A. Schematic representation of GFP modified with gp100-targeting sequences (gp100/GFP). 293T cells were transfected with plasmids coding for GFP, gp100 or gp100/GFP. The cells were permeabilized, stained with an anti-LAMP-1 conjugated with Alexa Fluor™-568 (in red) and analyzed by laser scanning confocal microscopy. White arrows revealed co-localization of gp100/GFP and LAMP-1. Results are representative of 5 independent experiments. B. Schematic representation of GFP modified with gp100-targeting sequences and minimal MHC class II and class I gp100 epitopes (gp/GFP+epit). 293T cells expressing HLA-DRβ1*0701 (DR7) or DRβ1*0401 (DR4) were transfected with plasmids encoding gp100 or gp/GFP+epit, and co-cultured with a gp100-specific CD4⁺ T cell clone (left panel). Plasmids encoding gp100 or gp/GFP+epit and HLA-A*0201 (A2) or A*0101 (A1) were co-transfected in 293T cells, and a gp100-specific CD8⁺ T cell clone was co-cultured with these transfected cells (right panel). Supernatants were harvested after 20 hours of co-culture, and IFN-γ secretion was measured by ELISA. Data are presented as a percentage of IFN-γ secretion compared to gp100, normalized to 100%. Data represents the average of 2 independent experiments. C. A melanoma cell line (MelFB) and HLA-DRβ1*0701⁺- or *0701-stimulated B lymphocytes (CD40-B) were electroporated with plasmids encoding gp100, gp/GFP+epit or tyrosinase, and were co-cultured with a gp100-specific CD4⁺ T cell clone. Data are representative of 2 independent experiments;

FIG. 7 shows the nucleotide (SEQ ID NO: 7), A) and amino acid (SEQ ID NO: 8), B) sequences of gp100 (GenBank accession Nos: S73003 and AAC60634, respectively). The coding sequence is indicated in bold in the nucleotide sequence;

FIG. 8 shows the structure of gp-M1 and gp-DKK1 constructs;

FIG. 9 shows expansion of T cells specific to gp-DKK1 in a T cell expansion experiment. After 14-20 days of expansion, individual microcultures were co-cultured with autologous APC expressing gp-DKK1 (□) or a negative control (▪). Supernatants were harvested after 20 hours of co-culture, and IFN-γ secretion was measured by ELISA. T cell lines were defined as being specific (indicated by boxes) when secretion with gp-DKK1 was higher than 50 pg/ml and twice the amount secreted when co-cultured with the control;

FIG. 10 shows expansion of T cells specific to gp-M1 in T cell expansion experiments. After 14-20 days of expansion, individual microcultures were co-cultured with autologous APC expressing gp-M1 (□) or a negative control (▪). Supernatants were harvested after 20-hour co-culture, and IFN-γ secretion was measured by ELISA. T cell lines were defined as being specific (indicated by boxes) when secretion with gp-M1 was higher than 50 pg/ml and twice the amount secreted when co-cultured with the control;

FIG. 11 shows the specificity of T cell lines #4 and #5 from donor #405. Lines #4 or #5 were co-cultured with autologous APC expressing gp-M1 pre-incubated or not with antibodies blocking presentation by either MHC class II (X-cl II) or class I (X-cl I). Alternatively, both lines were co-cultured with T2 cells pulsed with a known HLA-A2 M1 epitope. Supernatants were harvested after 20 hours of co-culture, and IFN-γ secretion was measured by ELISA;

FIG. 12 shows dose-dependent recognition of HLA-A*0201 gp100 peptides by a specific CD8⁺ T cell clone. HLA-A*0201⁺-stimulated B lymphocytes were pulsed with different concentrations of gp100₂₀₉₋₂₁₇, gp100₂₀₉₋₂₁₇/2M [qu'est ceci? 2M?] or FLU-M1₅₇₋₆₅ peptides. A gp100-specific CD8⁺ T cell clone was co-cultured with pulsed cells, and IFN-γ secretion was measured by ELISA; and [que peut-on conclure ici?]

FIG. 13 shows co-transfection of 293T cells with plasmids encoding GFP and gp100.

### DESCRIPTION OF ILLUSTRATIVE EMBODIMENTS

In the Examples described herein, Applicant has shown that a region located in the carboxy-terminal portion which comprises the putative gp100 transmembrane domain, and a region comprising the putative amino-terminal signal peptide of gp100 , are involved in MHC class II-mediated presentation of endogenous gp100 antigenic epitopes. Applicant has further demonstrated that the swapping of the putative transmembrane domain of gp100 with the putative transmembrane domain from another protein does not significantly affect MHC class II presentation of gp100 epitopes. Also, the addition of at least one of the above-mentioned regions/domains to other antigens induces/increases the presentation of epitopes derived from these antigens by MHC class II molecules.

Accordingly, in an aspect, the present invention provides a chimeric nucleic acid comprising:

(a) a first domain comprising a nucleic acid encoding signal peptide, or a fragment thereof retaining signal peptide activity; and

(b) a second domain comprising a nucleic acid encoding a polypeptide comprising (i) a transmembrane domain and (ii) an antigen or an epitope thereof,

wherein at least one of the signal peptide and the transmembrane domain is that of gp100 , and wherein said antigen or epitope is heterologous to at least one of said signal peptide and said transmembrane domain.

The present invention also relates to a vector comprising the above-mentioned chimeric nucleic acid. The present invention further relates to a cell comprising the above-mentioned chimeric nucleic acid or vector.

In another aspect, the present invention provides a polypeptide encoded by the above-mentioned chimeric nucleic acid.

In another aspect, the present invention provides a composition (e.g. a pharmaceutical composition or a vaccine composition) comprising the above-mentioned chimeric nucleic acid or the above-mentioned polypeptide, and a pharmaceutically acceptable carrier or excipient. In an embodiment, the above-mentioned composition further comprises an adjuvant.

The invention further provides a method for inducing or enhancing an immune response against an antigen in a subject, comprising administering to said subject a composition comprising a chimeric nucleic acid and an adjuvant, said chimeric nucleic acid comprising:

(a) a first domain comprising a nucleic acid encoding a signal peptide or a fragment thereof retaining signal peptide activity; and

(b) a second domain comprising a nucleic acid encoding a polypeptide comprising (i) a transmembrane domain and (ii) said antigen or an epitope thereof, wherein said antigen or epitope is heterologous to at least one of said signal peptide and said transmembrane domain.

In another aspect, the present invention provides a method for enhancing MHC class-II presentation of an antigen or an epitope thereof by a cell (e.g. an APC), comprising transfecting or transforming said cell with a nucleic acid comprising:

(a) a first domain comprising a nucleic acid encoding a signal peptide or a fragment thereof retaining signal peptide activity;

(b) a second domain comprising a nucleic acid encoding a polypeptide comprising (i) a transmembrane domain and (ii) said antigen or epitope thereof, wherein said antigenic epitope is heterologous to at least one of said signal peptide and said transmembrane domain.

In another aspect, the present invention provides a method for enhancing MHC class-II presentation of an antigenic epitope in a subject, comprising administering to said subject a composition comprising a chimeric nucleic acid and an adjuvant, said chimeric nucleic acid comprising:

(a) a first domain comprising a nucleic acid encoding a signal peptide or a fragment thereof retaining signal peptide activity; and

(b) a second domain comprising a nucleic acid encoding a polypeptide comprising (i) a transmembrane domain and (ii) said antigenic epitope, wherein said antigenic epitope is heterologous to at least one of said signal peptide and said transmembrane domain.

In yet another aspect, the present invention provides a method for decreasing or inhibiting an immune response against an antigen or an epitope thereof in a subject comprising administering to said subject a chimeric nucleic acid comprising;

(a) a first domain comprising a nucleic acid encoding a signal peptide or a fragment thereof retaining signal peptide activity;

(b) a second domain comprising a nucleic acid encoding a polypeptide comprising (i) a transmembrane domain and (ii) an antagonist of said antigen or epitope thereof, wherein said antagonist is heterologous to at least one of said signal peptide and said transmembrane domain.

In an embodiment, the above-mentioned first domain is N-terminal to said second domain. In another embodiment, the above-mentioned transmembrane domain is N-terminal to said antigen or epitope thereof or antagonist thereof. In another embodiment, the above-mentioned transmembrane domain is C-terminal to said antigen or epitope thereof or antagonist thereof. In another embodiment, the above-mentioned transmembrane domain is within said antigen or epitope thereof or antagonist thereof.

The terms “antigen” and “antigenic epitope” are very well-known in the art. An “antigen” generally refers to a molecule or a portion of a molecule capable of inducing an immune response (e.g., inducing the production of an antibody capable of binding to an epitope of that antigen and/or activating a T cells that has a T-cell receptor (TCR) recognizing an epitope of that antigen) in an animal. An antigen may have one or more epitope(s). “Antigenic epitope” or “epitope” are typically defined as the minimal structural unit of an antigen (the term “antigen” may thus refer to an “epitope”), recognizable for antibodies and lymphocyte antigenic receptors (e.g. T-cell receptors), that comes in contact with the antigen binding site of an antibody or the TCR. In the context of a T cell response, epitope refers to a peptide (typically between 8 to 20 amino acids) derived from an antigen which, when bound to an MHC molecule, is recognized by a T cell and induces its activation. The art teaches how to choose particularly antigenic determinants, how to increase the antigenicity of a peptide, molecule or the like, etc. The strength of an antigen is often referred to as the antigenicity or immunogenicity and relates to the property (which is often quantifiable) of eliciting or inducing an immune response. In an embodiment, the above-mentioned antigen is derived from a tumor (typically referred to as “tumor antigen” (TA) or tumor-associated antigen (TAA)). As used herein, the expression “tumor antigen” or “TA” refers to an antigen that is overexpressed in a tumor cell/tissue as compared to a corresponding normal cell/tissue. Overexpression can be, for example, an increase in expression of a given antigen in a tumor cell/tissue as compared to a normal cell/tissue, but also the expression of an antigen in a tumor cell/tissue that do not express it in a normal state (i.e. when the cell or tissue is not cancerous). For example, TA include known oncoproteins such as HER-2/Neu and c-myc, survival proteins such as survivin and lens epithelium-derived growth factor (LEDGF/p75), cell cycle regulatory proteins such as Cyclin B1, differentiation and cancer-testis antigens such as NY-ESO-1, colorectal cancer antigen such as carcinoembryonic antigen (CEA), most antigens of the MAGE family, melanA/MART-1, MUC1, Wilms' tumor protein (WT-1), STEAP and others (see Novellini et al, 2005. Cancer Immunol Immunother. 54(3):187-207 for a list of known TA). In a further embodiment, the above-mentioned antigen is DKK1 (Dickkopf-1).

In another embodiment, the above-mentioned antigen is derived from a pathogen (e.g., a bacteria, a fungus, a virus, a parasite). In a further embodiment, the above-mentioned antigen is derived from a virus (typically referred to as “viral antigen”). Viral antigens include, but are not limited to, HIV proteins such as HIV gag proteins (including, but not limited to, membrane anchoring (MA) protein, core capsid (CA) protein and nucleocapsid (NC) protein), HIV polymerase, influenza virus matrix (M1) protein and influenza virus nucleocapsid (NP) protein, hepatitis B surface antigen (HBsAg), hepatitis B core protein (HBcAg), hepatitis e protein (HBeAg), hepatitis B DNA polymerase, hepatitis C antigens, and the like. Other examples of antigen polypeptides are group- or sub-group specific antigens, which are known for a number of infectious agents, including, but not limited to, adenovirus, herpes simplex virus, papilloma virus, respiratory syncytial virus and poxviruses. In a further embodiment, the above-mentioned viral antigen is influenza virus M1 matrix protein.

“Signal peptide” (also referred to as “leader peptide”) as used herein refers to a polypeptide (typically from about 3 to about 60 amino acids) that direct the post-translational transport of a polypeptide into the endoplasmic reticulum (ER). These signal peptides are usually found at the amino terminus of secreted/transmembrane proteins. Signal peptides from diverse organisms are well known in the art. Several peptide signals are known. For instance, SPdb, a signal peptide database lists a number of useful signal peptides (Choo K H, et al., 2005. BMC Bioinformatics 6:249).

In an embodiment, the above-mentioned signal peptide is the first 20 amino acids of gp100 , or a fragment or variant thereof retaining signal peptide activity/function (e.g., the activity/function of directing the post-translational transport of a polypeptide into the endoplasmic reticulum).

“Transmembrane domain” as used herein refers to a domain of a protein, typically comprising alpha helice(s), which permits the anchoring of the proteins into a membrane (e.g. a cell membrane or an organelle membrane). Transmembrane domains from several proteins have been described and are thus well known in the art. TMbase™ is a database of transmembrane proteins (Hofmann K. and Stoffel W. 1993. TMBASE-A database of membrane spanning protein segments Biol. Chem. Hoppe-Seyler 374: 166) with their helical membrane-spanning (TM) domain. Without being so limited, they include that derived from the human angiotensin converting enzyme-2 (ACE2 i.e. the SARS-Corona Virus receptor), Lamp-1 and LDLR. Furthermore, a transmembrane domain may be an artificial sequence of hydrophobic amino acids that permits the anchoring of proteins across a membrane, and may thus be synthesized.

In an embodiment, the above-mentioned transmembrane domain is the transmembrane domain of gp100 or CD8. In another embodiment, the above-mentioned transmembrane domain comprises the sequence of SEQ ID NO:1 or 4. In a further embodiment, the above-mentioned nucleic acid encoding a transmembrane domain comprises the sequence of SEQ ID NO:10 or 13.

In another embodiment, the above-mentioned polypeptide comprises the sequence of SEQ ID NO:2. In yet another embodiment, the above-mentioned polypeptide further comprises the sequence of SEQ ID NO:5. In a further embodiment, said transmembrane domain is that of CD8 and the polypeptide further comprises at the C-terminal of the transmembrane domain the sequence of SEQ ID NO:5. In an embodiment, the above-mentioned nucleic acid encoding a polypeptide comprises the sequence of SEQ ID NO:11. In a further embodiment, the above-mentioned nucleic acid encoding a polypeptide further comprises the sequence of SEQ ID NO:14.

In an embodiment, the above-mentioned immune response is a T-cell response. In a further embodiment, the above-mentioned T-cell response is a CD4⁺ T cell response.

As used herein, the term “heterologous”, when referring to two nucleic acid/polypeptide domains or fragments, indicates that the two domains or fragments originate (or are derived) from different nucleic acids/proteins (e.g. a first domain from polypeptide A with a second domain from polypeptide B).

Within the context of the invention are polypeptides and nucleic acids which are homologous to or substantially identical with, based on sequence, a chimeric nucleic acid or polypeptide of the invention and retain the relevant function.

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to another sequence if the two sequences are substantially identical and the functional activity of the sequences is conserved (as used herein, the term ‘homologous’ does not infer evolutionary relatedness). Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95% identity. As used herein, a given percentage of homology between sequences denotes the degree of sequence identity in optimally aligned sequences. An “unrelated” or “non-homologous” sequence shares less than 40% identity, though preferably less than about 25% identity, with a nucleic acid sequence of the present invention.

Substantially complementary nucleic acids are nucleic acids in which the complement of one molecule is substantially identical to the other molecule. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). Generally, stringent conditions are selected to be about 5° C. lower than the thermal melting point for the specific sequence at a defined ionic strength and pH.

The term “vector” is commonly known in the art and defines e.g., a plasmid DNA, phage DNA, viral DNA and the like, which can serve as a DNA vehicle into which DNA of the present invention can be cloned. Numerous types of vectors exist and are well known in the art. In a particular embodiment, the vector is a viral vector which can introduce a molecule, e.g., a chimeric nucleic acid of the invention, in a cell or in a living organism.

Various genes and nucleic acid sequences of the invention may be recombinant sequences. The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule which is expressed using a recombinant nucleic acid construct created by means of molecular biological techniques. The term “recombinant” when made in reference to genetic composition refers to a gamete or progeny or cell or genome with new combinations of alleles that did not occur in the parental genomes. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as ‘recombinant’ therefore indicates that the nucleic acid molecule has been manipulated using genetic engineering, i.e. by human intervention. Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation or transfection into a host. The cloned gene (inserted sequence) is usually placed under the control of control element or transcriptionally regulatory sequences such as promoter sequences. The placing of a cloned gene under such control sequences is often referred to as being operably linked to control elements or sequences.

A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since for example enhancers generally function when separated from the promoters by several kilobases and intronic sequences may be of variable lengths, some polynucleotide elements may be operably-linked but not contiguous. “Transcriptional regulatory element” is a generic term that refers to DNA sequences, such as initiation and termination signals, enhancers, and promoters, splicing signals, polyadenylation signals which induce or control transcription of protein coding sequences with which they are operably-linked.

Operably-linked sequences may also include two segments that are transcribed onto the same RNA transcript. Thus, two sequences, such as a promoter and reporter sequence are operably linked if transcription commencing in the promoter will produce an RNA transcript of the reporter sequence. In order to be “operably-linked” it is not necessary that two sequences be immediately adjacent to one another.

Expression control sequences will vary depending on whether the vector is designed to express the operably-linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e. g. SDS gel electrophoresis, gel filtration, centrifugation, ion exchange chromatography, etc.). In addition, the protein of interest can be purified via affinity chromatography using polyclonal or monoclonal antibodies or a specific ligand. The purified protein can be used for therapeutic applications. Prokaryotically expressed eukaryotic proteins are often not glycosylated.

The DNA (or RNA) construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is preferably bound at its 3′ terminus by the transcription initiation site and extends upstream (5′ direction) to include the minimum number of bases or elements necessary to initiate transcription at levels detectable above background.

Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain −10 and −35 consensus sequences, which serve to initiate transcription and the transcript products contain Shine-Dalgarno sequences, which serve as ribosome binding sequences during translation initiation. Non-limiting examples of vectors which can be used in accordance with the present invention include adenoviral vectors, poxviral vectors, VSV-derived vectors and retroviral vectors. Such vectors and others are well known in the art.

As used herein, the designation “functional derivative” or “functional variant” denotes, in the context of a functional derivative of a sequence whether a nucleic acid or amino acid sequence, a molecule that retains a biological activity (either function or structural) that is substantially similar to that of the original sequence (e.g. a signal peptide activity). This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid generally has chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

Thus, the term “variant” refers herein to a protein or nucleic acid molecule which is substantially similar in structure and biological activity to the protein or nucleic acid of the present invention but is not limited to a variant which retains all of the biological activities of the parental protein, for example. The functional derivatives of the present invention can be synthesized chemically or produced through recombinant DNA technology. All these methods are well known in the art.

For certainty, the sequences and polypeptides useful to practice the invention include without being limited thereto mutants, homologs, subtypes, alleles and the like. It will be clear to the person of ordinary skill that whether an interaction domain of the present invention, variant, derivative, or fragment thereof retains its function in binding to its partner can be readily determined by using the teachings and assays of the present invention and the general teachings of the art.

Also within the context of the present invention is the in vivo administration of a nucleic acid or a vector of the invention to a subject, such as gene therapy and/or immunization/vaccination methods.

Nucleic acids may be delivered to cells in vivo using methods such as direct injection of DNA, receptor-mediated DNA uptake, viral-mediated transfection or non-viral transfection and lipid-based transfection, all of which may involve the use of gene therapy vectors. Direct injection has been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo may be used. Such an apparatus may be commercially available (e.g., from BioRad). Naked DNA may also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor may facilitate uptake of the DNA by receptor-mediated endocytosis. A DNA-ligand complex linked to adenovirus capsids which disrupt endosomes, thereby releasing material into the cytoplasm, may be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126).

Defective retroviruses are well characterized for use as gene therapy vectors (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Recombinant ALVAC virus are also known in the art (Godelaine, D et al., 2003. J. Immunol. 171: 4893-4897; Karanikas, V. et al., 2003. J. Immunol. 171: 4898-4904.) Examples of suitable packaging virus lines include .psi.Crip, .psi.Cre, .psi.2 and .psi.Am. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Adeno-associated virus (AAV) may be used as a gene therapy vector for delivery of DNA for gene therapy purposes. AAV is a naturally occurring defective virus that requires another virus, such as an adenovirus or a herpes virus, as a helper virus for efficient replication and a productive life cycle (Muzyczka et al., 1992. Curr. Topics in Micro. and Immunol. 158:97-129). AAV may be used to integrate DNA into non-dividing cells (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). An AAV vector such as that described in Tratschin et al. (1985) Mol. Cell. Biol. 5:3251-3260 may be used to introduce DNA into cells (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). Lentiviral gene therapy vectors may also be adapted for use in the invention.

Also within the scope of the invention are cells (e.g. host cells) transfected or transformed with the chimeric nucleic acid or the vector of the invention. Methods for transforming/transfecting host cells with nucleic acids/vectors are well-known in the art and depend on the host system selected as described in Ausubel et al. (Ausubel et al., Current Protocols in Molecular Biology, John Wiley & Sons Inc., 1994). The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Host cells transfected or transformed with the chimeric nucleic acid or vector of the invention can be used as a vaccine (e.g., autologous cell vaccine) in order to induce or increase an immune response against an antigenic epitope in a subject. For example, host cells (e.g., APCs, dendritic cells) may be removed from a subject (e.g. a cancer patient, or a subject infected with a pathogen [or susceptible of being infected by a pathogen]), transfected or transformed in accordance with the present invention and re-administered to the patient. Of course, known steps for further cultivating or modifying these cells could be carried-out prior to re-injecting/transplanting them into a subject. For example, cytokines/chemokines or mitogens or molecules could be added to the culture medium. DCs are conveniently categorized as “immature” and “mature” cells and allow for an easy discrimination of two well-characterized phenotypes. Immature DCs are CD11c⁺, MHC class II⁺, CD86⁺, CD80^{low}, CD14⁻ and CD83⁻, and fail to secrete IL-12. Following proper stimulation/maturation with a combination of CD40L and lipopolysaccharides, or CD40L and poly I:C for example, CD80 and CD83 increase and they secrete high level of IL-12 (Lapointe, R. et al., 2000. Eur J Immunol 30: 3291-3298). In order to optimize their capacity of stimulating TA-specific T cells, DCs may need to be properly activated or matured (Banchereau, J. and Palucka, A. K., 2005. Nature Reviews Immunology 5: 296-306). Matured-TA-expressing DCs could be a means of expanding TA-specific T lymphocytes dedicated for adoptive transfer.

In accordance with another embodiment of the present invention, T cells (e.g., CD4⁺ T cells) may be removed from a subject (e.g. cancer patient, or virally affected patient [or susceptible of being infected by a virus]), activated in accordance with the present invention (e.g., by contacting them with a host cells transfected with a chimeric nucleic acid of the invention) and re-administered to the patient. Of course, known steps for further cultivating or proliferating these T cells could be carried-out prior to re-injecting them into a subject. For example, cytokines or other mitogens or molecules could be added to the culture medium.

In an embodiment, the above-mentioned cells are APCs (e.g., dendritic cells (DC), activated B cells, activated macrophages). In a further embodiment, the above-mentioned APCs are dendritic cells.

A chimeric nucleic acid of the invention can also be useful as a vaccine. There are two major routes, either using a viral or bacterial host as gene delivery vehicle (live vaccine vector) or administering the gene in a free form, e.g., inserted into a plasmid. Therapeutic or prophylactic efficacy of a polynucleotide of the invention is evaluated as described below.

“Vaccine” as used herein refers to a composition or formulation comprising one or more polypeptides/peptides of the invention, or a vaccine vector of the invention. Vaccination methods for treating or preventing an infection or a disease in an animal (e.g., a mammal, such as a human) comprises use of a vaccine or vaccine vector of the invention to be administered by any conventional route known the vaccine field, in such as to a mucosal (e.g., ocular, intranasal, pulmonary, oral, gastric, intestinal, rectal, vaginal, or urinary tract) surface, via a parenteral (e.g., subcutaneous, intradermal, intramuscular, intravenous, or intraperitoneal) route, or topical administration (e.g. via a patch). The choice of administration route depends upon a number of parameters, such as the adjuvant associated with the polypeptide. If a mucosal adjuvant is used, the intranasal or oral route is preferred. If a lipid formulation or an aluminum compound is used, the parenteral route is preferred with the sub-cutaneous or intramuscular route being most preferred. The choice also depends upon the nature of the vaccine agent.

Accordingly, a further aspect of the invention provides (i) a vaccine vector such as an adenovirus, containing a chimeric nucleic acid molecule of the invention, placed under the control of elements required for expression; (ii) a composition of matter comprising a vaccine vector of the invention, together with a diluent or carrier; specifically (iii) a pharmaceutical composition containing a therapeutically or prophylactically effective amount of a vaccine vector of the invention; (iv) an immunogenic composition (e.g. a vaccine) comprising the above-mentioned vaccine vector or composition together with an adjuvant; (v) a method for inducing or enhancing an immune response (e.g. a CD4⁺ or “helper” T cell response) against an antigen/antigenic epitope (e.g., a tumor antigen, an antigen from a pathogen such as a bacteria or virus) in an animal (e.g., a human; alternatively, the method can be used in veterinary applications for treating or preventing a disease (e.g., tumor growth or infection) in non-human animals), which involves administering to the mammal an immunogenically effective amount of a vaccine vector of the invention to elicit a protective or therapeutic immune response; and particularly, (vi) a method for preventing and/or treating a disease (e.g., cancer, infectious disease), which involves administering a prophylactic or therapeutic amount of a vaccine vector, or a composition, of the invention to an individual having, or at risk of (e.g., susceptible to) developing, the disease. Additionally, the invention further provides a use of a vaccine vector of the invention in the preparation of a medicament for preventing and/or treating a disease.

As used herein, “prevention” and/or “treatment” is an approach for obtaining beneficial or desired results, including clinical results. For purposes of this invention, beneficial or desired clinical results include, but are not limited to, (i) prevention, that is, causing the clinical symptoms not to develop, e.g., preventing disease/infection from occurring and/or developing to a harmful state; (ii) alleviation or amelioration of one or more symptoms, (iii) diminishment of extent of disease, (iv) stabilizing (i.e., not worsening) state of disease, (v) preventing spread of disease, (vi) delay or slowing of disease progression, (vii) amelioration or palliation of the disease state, and (viii) remission (whether partial or total), whether detectable or undetectable. “Treatment” can also mean prolonging survival as compared to expected survival if not receiving treatment.

In an embodiment, the above-mentioned treatment/method comprises the use/administration of more than one (i.e. a combination of) active/therapeutic agent. The combination of prophylactic/therapeutic agents and/or compositions of the present invention may be administered or co-administered (e.g., consecutively, simultaneously, at different times) in any conventional dosage form. Co-administration in the context of the present invention refers to the administration of more than one therapeutic in the course of a coordinated treatment to achieve an improved clinical outcome. Such co-administration may also be coextensive, that is, occurring during overlapping periods of time. For example, a first agent may be administered to a patient before, concomitantly, before and after, or after a second active agent is administered. The agents may in an embodiment be combined/formulated in a single composition and thus administered at the same time. In an embodiment, the one or more active agent(s) of the present invention (e.g. a chimeric nucleic acid or encoded polypeptide) is used/administered in combination with one or more agent(s) currently used to prevent or treat the disorder in question (e.g., a vaccine such as an influenza vaccine, an anticancer or antimicrobial agent).

As used herein, the terminology “immune response” refers to any reaction of the immune system against a foreign biological material (i.e. antigen). As used herein the terminology “immune system” refers to the collection of organs and tissues and cells involved in the adaptive defense of a body against foreign biological material. It may be broken down into the adaptive immune system, composed of four lymphoid organs (thymus, lymph nodes, spleen and submucosal lymphoid nodules) and the group motile cells that are involved in the body's defense against foreign bodies. Without being so limited, immune response include in vivo or ex vivo “T lymphocytes activation” in an antigen-specific manner by triggering of the TCR, as illustrated by T cell proliferation, or secretion of an array of cytokines such as but not limited to GM-CSF, TNF-α, IFN-γ, IL-2, IL-4 and IL-10, or evidence of cytolytic activity such as but not limited to secretion of perforin, granzyme family members, or migration of CD107a (LAMP-1) to the cell surface or any functional assay demonstrating lysis of a relevant target. Upregulation of some surface or intracellular molecules can also serve as T cell activation markers, such as but not limited to CTLA-4, CD25 (high affinity IL-2 receptor) Ki-67, or MHC class II molecules.

As used herein, a vaccine vector expresses one or several polypeptides or derivatives of the invention. The vaccine vector may express additionally a cytokine, such as interleukin-2 (IL-2) or interleukin-12 (IL-12), or co-stimulatory molecules, which enhances the immune response (adjuvant effect). It is understood that each of the components to be expressed is placed under the control of elements required for expression in a mammalian cell.

The composition comprising a polypeptide or vaccine vector of the present invention may further contain an adjuvant. A number of adjuvants are known to those skilled in the art. Adjuvants for parenteral administration include aluminum compounds, such as aluminum hydroxide, aluminum phosphate, and aluminum hydroxy phosphate. The antigen is precipitated with, or adsorbed onto, the aluminum compound according to standard protocols. Other adjuvants, such as RIBI (ImmunoChem, Hamilton, Mont.), are used in parenteral administration.

Adjuvants for mucosal administration include bacterial toxins, e.g., the cholera toxin (CT), the E. coli heat-labile toxin (LT), the Clostridium difficile toxin A and the pertussis toxin (PT), or combinations, subunits, toxoids, or mutants thereof such as a purified preparation of native cholera toxin subunit B (CTB). Fragments, homologs, derivatives, and fusions to any of these toxins are also suitable, provided that they retain adjuvant activity. Preferably, a mutant having reduced toxicity is used. Suitable mutants are described, e.g., in WO 95/17211 (Arg-7-Lys CT mutant), WO 96/06627 (Arg-192-Gly LT mutant), and WO 95/34323 (Arg-9-Lys and Glu-129-Gly PT mutant). Additional LT mutants that are used in the methods and compositions of the invention include, e.g., Ser-63-Lys, Ala-69Gly, Glu-110-Asp, and Glu-112-Asp mutants. Other adjuvants, such as a bacterial monophosphoryl lipid A (MPLA) of, e.g., E. coli, Salmonella minnesota, Salmonella typhimurium, or Shigella flexneri; saponins, or polylactide glycolide (PLGA) microspheres, is also be used in mucosal administration.

Adjuvants useful for both mucosal and parenteral administrations include polyphosphazene (WO 95/02415), DC-chol (3 b-(N-(N′,N′-dimethyl aminomethane)-carbamoyl) cholesterol; U.S. Pat. No. 5,283,185 and WO 96/14831) and QS-21 (WO 88/09336).

Treatment is achieved in a single dose or repeated as necessary at intervals, as can be determined readily by one skilled in the art. For example, a priming dose is followed by three booster doses at weekly or monthly intervals. An appropriate dose depends on various parameters including the recipient (e.g., adult or infant), the particular vaccine antigen, the route and frequency of administration, the presence/absence or type of adjuvant, and the desired effect (e.g., protection and/or treatment), as can be determined by one skilled in the art.

Any pharmaceutical composition of the invention containing a chimeric polypeptide, a polypeptide derivative or a chimeric nucleic acid of the invention, is manufactured in a conventional manner. In particular, it is formulated with a pharmaceutically acceptable diluent or carrier, e.g., water or a saline solution such as phosphate buffer saline. In general, a diluent or carrier is selected on the basis of the mode and route of administration, and standard pharmaceutical practice. As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Suitable pharmaceutical carriers or diluents, as well as pharmaceutical necessities for their use in pharmaceutical formulations, are described in Remington's Pharmaceutical Sciences, a standard reference text in this field and in the USP/NF. Except insofar as any conventional media or agent is incompatible with the active compound, use thereof in the pharmaceutical compositions of the invention is contemplated. Supplementary active compounds can also be incorporated into the compositions.

Compositions within the scope of the present invention should contain the active agent (e.g. chimeric nucleic acid and/or polypeptide, cells (e.g. APCs) in an amount effective to achieve the desired increase in immunogenicity of an antigen or antigenic epitope (e.g. increase in epitope-specific T cell activation) while avoiding adverse side effects. Typically, the chimeric nucleic acids in accordance with the present invention can be administered to mammals (e.g., humans) in doses ranging from 0.001 to 50 mg per kg of body weight per day of the mammal which is treated. Pharmaceutically acceptable preparations and salts of the active agent are within the scope of the present invention and are well known in the art (Remington's Pharmaceutical Science, 16^{th} ed., Mack ed.). The invention therefore further provides a composition comprising an active agent and a pharmaceutically acceptable carrier. For the administration of polypeptides, antagonists, agonists and the like, the amount administered should be chosen so as to avoid adverse side effects. The dosage will be adapted by the clinician in accordance with conventional factors such as the extent of the disease and different parameters from the subject. The composition of the present invention may also comprise one or more additional active agent(s) (e.g. another vaccine such as an influenza vaccine, an antimicrobial or anticancer agent, a modulator of the immune response).

A chimeric nucleic acid of the present invention may alternatively be used in a method for decreasing or inhibiting the immune response against an antigen and/or an antigenic epitope. Such decrease or inhibition of the immune response may be particularly useful for the treatment of diseases in which an inappropriate and/or undesirable and/or excessive immune response is involved (e.g. autoimmune diseases, inflammatory diseases, transplant rejection, allergies). Examples of autoimmune diseases include but are not limited to, Addision's disease, alopecia greata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others. Examples of antigens and antigenic epitopes involved in these diseases have been described and are well known by those of skill in the art (See, for example, US Patent Application No. 2006/0257420).

Such a decrease or inhibition of the immune response against an antigen or epitope may be achieved by contacting T cells from a subject with one or more antagonist(s) of the antigen. For example, antagonist peptides/epitopes (also sometimes referred to as “altered peptide ligands”) are typically obtained by mutating or modifying one or more specific residues in the wild-type peptide/epitope. T cells exposed to an antagonist peptide become anergic or tolerant to the wild-type peptide. As used herein, the term “anergy” or “tolerance” includes refractivity to activating receptor-mediated stimulation. Such refractivity is generally antigen-specific and persists after exposure to the tolerizing antigen has ceased. For example, anergy in T cells is generally characterized by lack of cytokine production, e.g., IL-2.

As used herein, the term “subject” or “patient” generally refers to both humans and other animals, such as domestic animals. In an embodiment, the above-mentioned animal is a pet animal (e.g., a dog, a cat). In another embodiment, the above-mentioned animal is a livestock (e.g., bovine, swine, equine, sheep, poultry such as chicken). In another embodiment, the above-mentioned animal is a mammal. In a further embodiment, the above-mentioned mammal is a human. As such, the above-mentioned compounds and methods may be used for both human therapy and veterinary applications, for example for the vaccination of animals.

The present invention is illustrated in further details by the following non-limiting examples.

### EXAMPLES

### Example 1

Material and Methods

### Media and Cell Culture.

Complete medium consisted of AIM-V medium (Invitrogen; Carlsbad, Calif.) supplemented with 5% human AB serum (heat-inactivated; Gemini Bio-Products; Calabasas, Calif.), 2 mM L-glutamine, 100 U/ml penicillin/streptomycin and 10 mg/ml gentamicin (all from Invitrogen). A gp100-specific, HLA-DRβ1*0701-restricted CD4⁺ T cell clone and an HLA-A*0201-restricted CD8⁺ T cell clone were cultured as described previously (Lapointe R, et al., 2001. J. Immunol. 167:4758-4764; Dudley et al., 1999. J. Immunother. 22:288-298) in complete medium supplemented with 300 IU/ml recombinant human Interleukin (IL)-2 (Chiron; Emeryville, Calif.).

CD40-stimulated B lymphocytes (CD40-B) were cultured as described previously (Lapointe et al., 2003. Cancer Res. 63:2836-2843) in Iscove's Modified Dulbecco's Medium (Invitrogen; and Wisent; St-Bruno, Quebec, Canada) supplemented with 10% human serum (heat-inactivated, prepared from normal donors), 2 mM L-glutamine, 100 U/ml penicillin/streptomycin, 10 mg/ml gentamicin, 500 ng/ml of a soluble trimeric CD40L (Immunex Corporation; Seatle, Wash.) and 500 U/ml recombinant human IL-4 (Peprotech; Rocky Hill, N.J.).

HEK-293T cells expressing HLA-DRβ1*0701 or DRβ1*0401, kindly provided by Dr. Paul F. Robbins and Dr. Suzanne L. Topalian (NCI/NIH; Bethesda, Md.), and HEK-293T cells expressing HLA-A*0201 were cultured in RPMI 1640 medium (Invitrogen and Wisent) supplemented with 10% heat-inactivated FBS (Invitrogen and Wisent), 2 mM L-glutamine, 100 U/ml penicillin/streptomycin and 10 mg/ml gentamicin (Lepage, S. and Lapointe, R., 2006. Cancer Research. 66(4):2423-32).

The melanoma cell line MelFB, which was immuno-selected for the absence of gp100 and MART-1, was transduced by retroviral vectors encoding gp100 or green fluorescent protein (GFP), as described previously (Lapointe R, et al., 2001. J. Immunol. 167:4758-4764). Melanoma cell lines 1087mel, 624.38mel, 624.38mel-CIITA, 1088mel, 1102mel, 1300mel, 397mel, 553mel and SK23mel were established at the Surgery Branch (NCI/NIH) (Parkhurst, M R et al., 2004. J. Immunother. 27: 79-91; Milani, V. et al., 2005. International Immunology 17: 257-268; Kawakami et al., 1998. J Immunol. 161(12):6985-92).

Breast tumor cell lines MCF-7 and MDA231 were obtained from the American Type Culture Collection (ATCC; Manassas, Va.). All tumor cell lines were cultured in RPMI 1640 medium supplemented with 10% heat-inactivated FBS, 2 mM L-glutamine, 100 U/ml penicillin/streptomycin and 10 mg/ml gentamicin.

### Gp100 Mutants and Other Plasmids

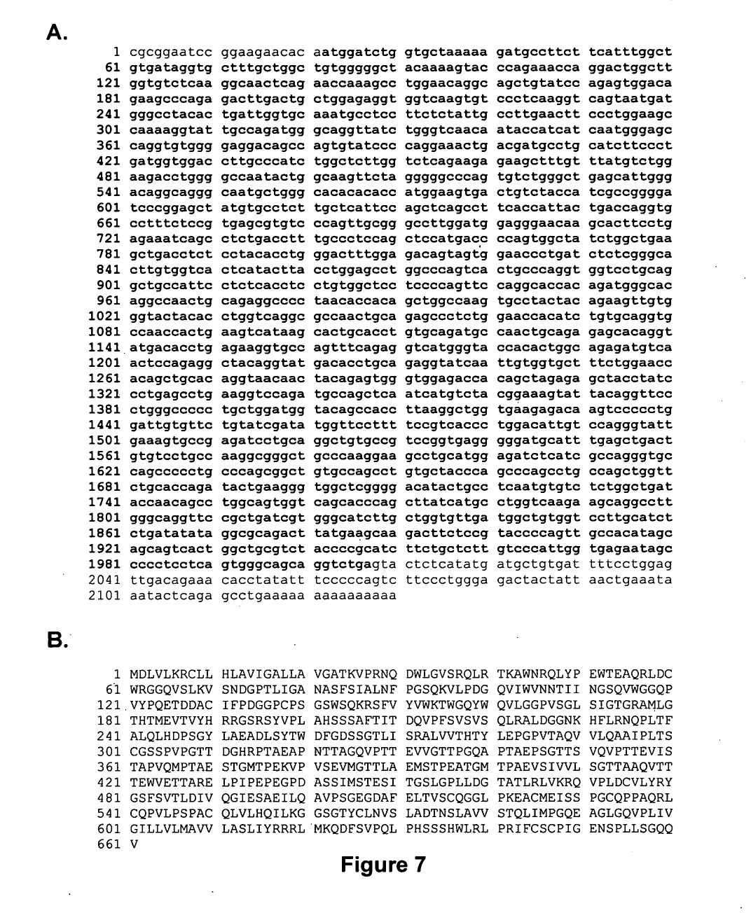

Plasmids encoding HLA-A*0201 and A*0101 (pcDNA-A2 and CLNCx-A1 respectively), kindly supplied by Dr. Paul F. Robbins, were cloned from HLA-typed patients, at the NIH. Plasmid encoding gp100 (pcDNA-gp100) also was provided by Dr. Paul F. Robbins (NCI/NIH) (Lapointe et al., J Immunol 167:4758-4764). Gp100 nucleotide and amino acid sequences (SEQ ID NOs: 7 and 8, respectively) are available from Genbank (accession numbers S73003 and AAC60634, respectively) and are provided in FIG. 7.

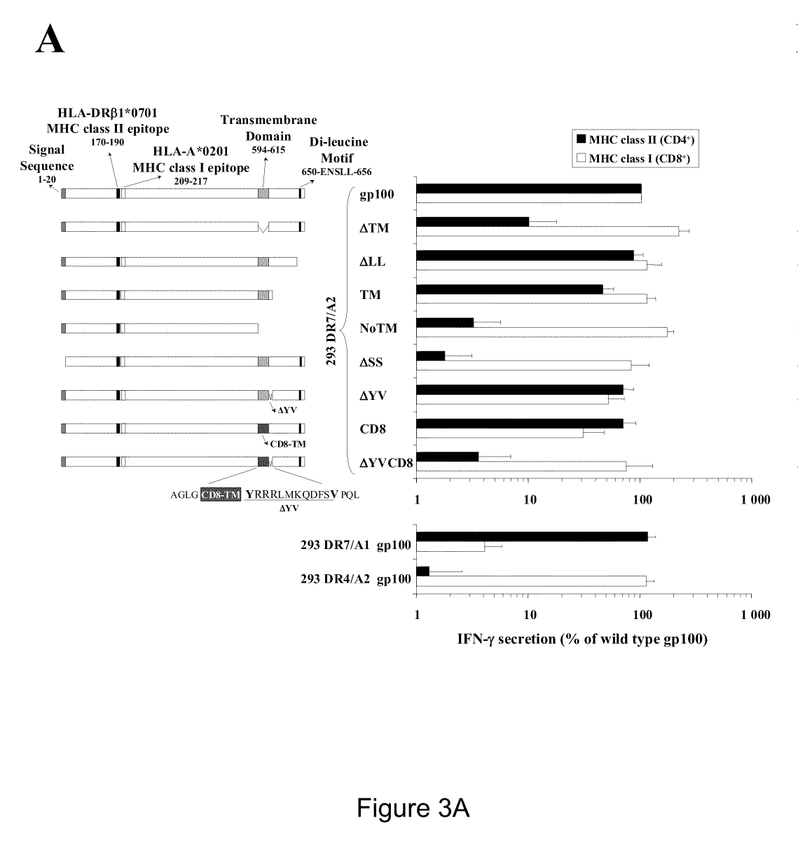

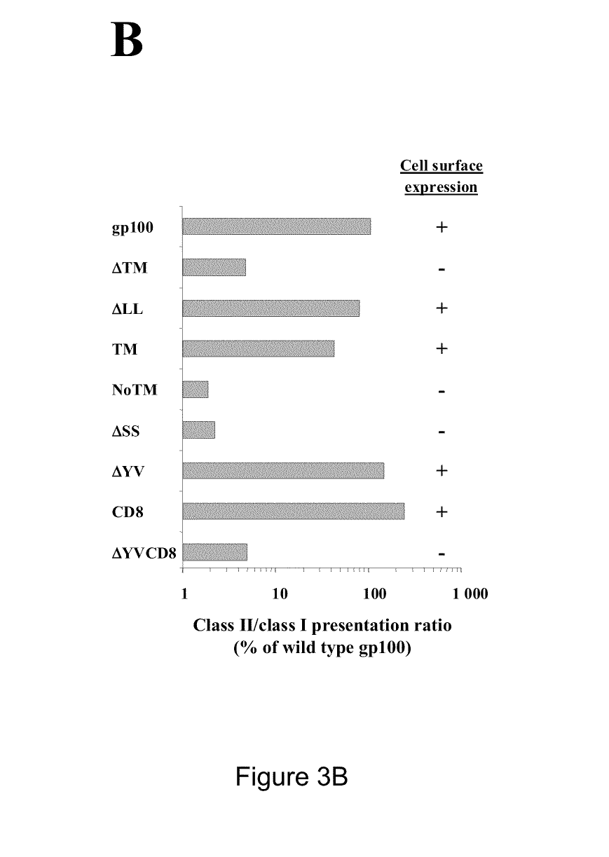

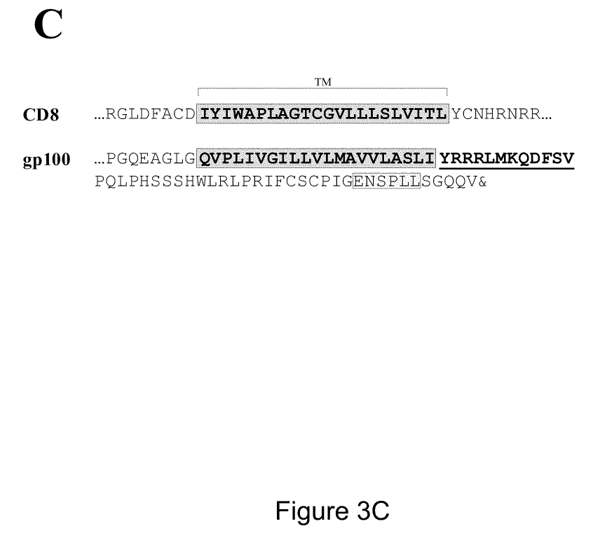

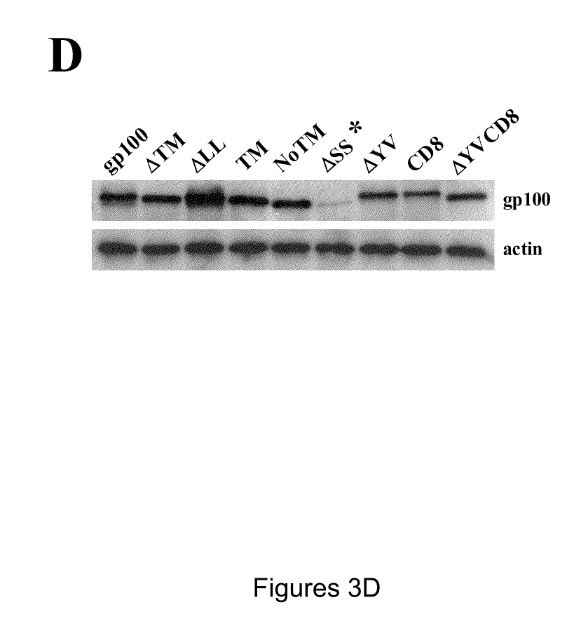

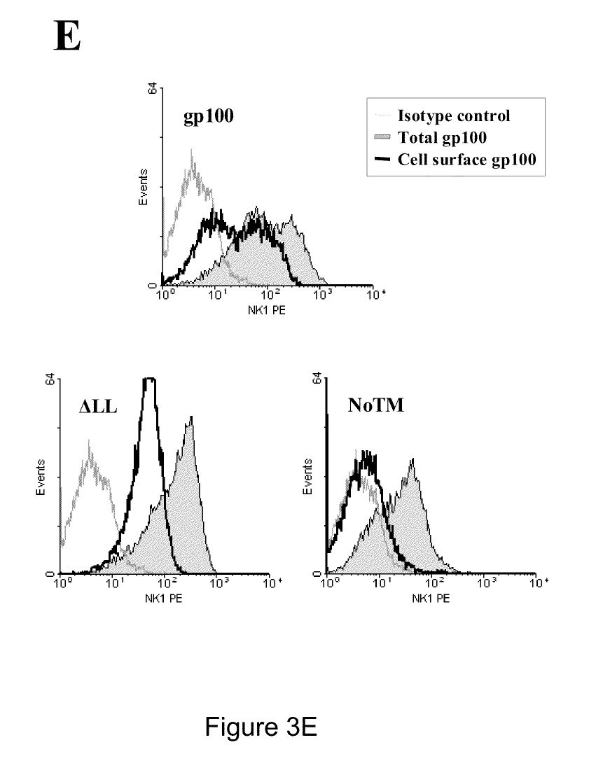

Plasmids encoding the different gp100 mutants, deleted at the carboxy-terminus or amino-terminus (presented in FIG. 3A (left panel)), were prepared by PCR from the wild-type sequence, cloned into pcDNA3.1, and their sequences were confirmed by sequencing. To generate pcDNA-gp100ΔTM, the region corresponding to residues 595 to 615 of gp100 (QVPLIVGILLVLMAVVLASLI; SEQ ID NO:1), which corresponds to the putative transmembrane domain, was deleted. To generate pcDNA-gp100ΔLL, the C-terminal portion of gp100, starting from residue 650 to the C-terminal end, was deleted. To generate pcDNA-gp100TM, the C-terminal portion of gp100, starting from residue 616 to the C-terminal end, was deleted. PcDNA-gp100NoTM was generated by deleting the C-terminal region of gp100, from residue 595 to the C-terminal end (QVPLIVGILLVLMAVVLASLIYRRRLMKQDFSVPQLPHSSSHWLRLPRIFCSCPI GENSPLLSGQQV; SEQ ID NO:2). To generate PcDNA-gp100ΔSS, residues 1 to 20 from gp100 (MDLVLKRCLLHLAVIGALLA; SEQ ID NO:3) were deleted. PcDNA-gp100ΔYV was generated by deleting residues 616 to 627 of gp100 (YRRRLMKQDFSV; SEQ ID NO:5). In pcDNA-gp100CD8, the putative transmembrane domain gp100 (residues 595 to 615) was swapped with the putative transmembrane domain of CD8α (residues 183 to 204 of CD8α; IYIWAPLAGTCGVLLLSLVITL; SEQ ID NO:4). PcDNA-gp100ΔYVCD8 is similar to pcDNA-gp100CD8, except that residues 616 to 627 of gp100 (YRRRLMKQDFSV; SEQ ID NO:5) were also removed.

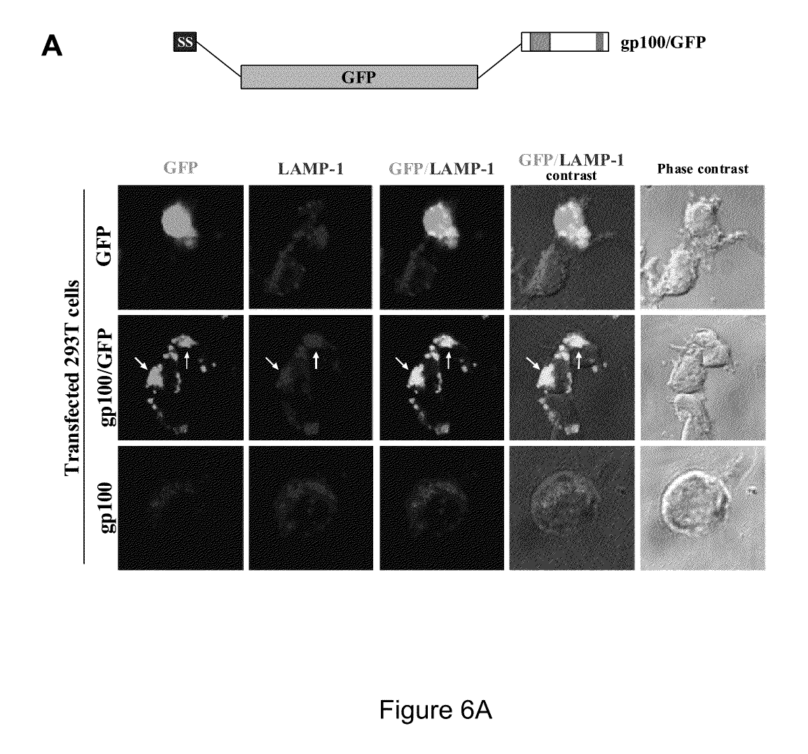

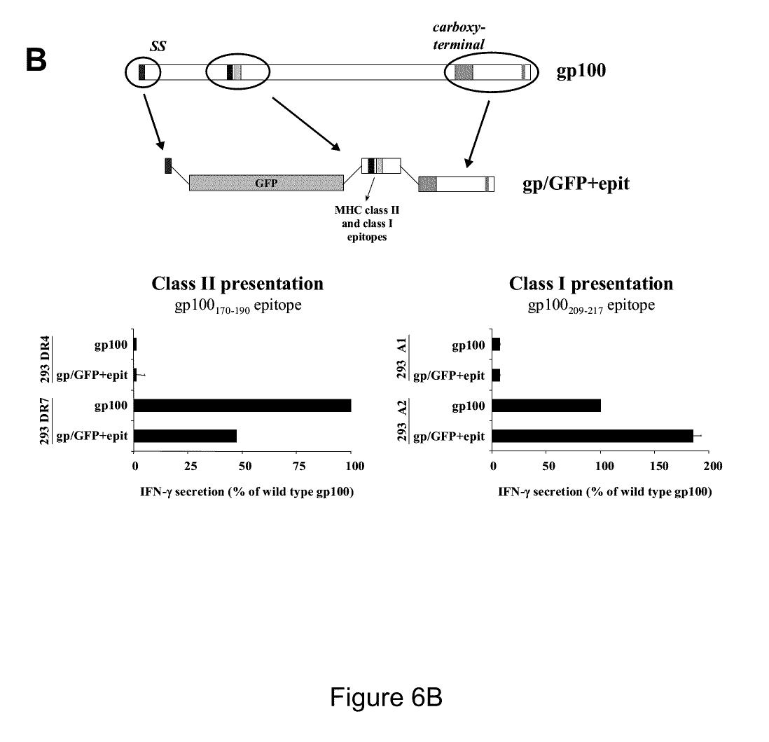

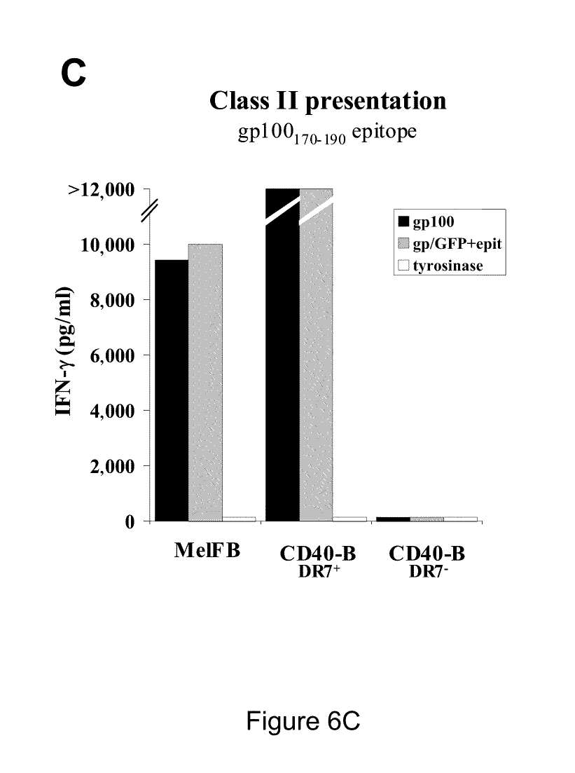

To generate the pcDNA-gp100/GFP construct (presented in Figure 6A), the entire GFP sequence, from which the first (N-terminal) methionine was replaced by a valine, was cloned between residues 20 and 595 of gp100 (i.e. residues 21 to 594 of gp100 were replaced by the above-identified sequence of GFP). In pcDNA-gp/GFP+epit (presented in FIG. 6B), residues 150 to 225 from gp100, which corresponded to minimal MHC class II and class I epitopes, were inserted after the GFP sequence.

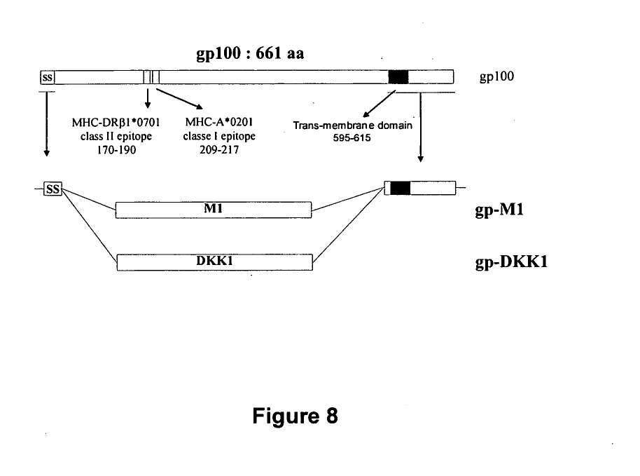

To generate the gp100-M1 and gp100/-DKK1 construct (presented in FIG. 8), the entire M1 and DKK1 sequences were each cloned between residues 20 and 578 of gp100 (i.e. residues 21 to 577 of gp100 were replaced by the above-identified sequence of M1 or DKK1). Residues 578-661 of gp100: AVV STQLIMPGQE AGLGQVPLIV GILLVLMAVV LASLIYRRRL MKQDFSVPQL PHSSSHWLRL PRIFCSCPIG ENSPLLSGQQ V (SEQ ID NO:6).

### Cell Transfection and APC-Pulsing

The day before transfection, cells were plated at 5×10⁵ cells/well in 6-well plates in order to reach about 50-90% confluence on the day of transfection. Cells were transiently transfected employing Lipofectamine™ Plus Reagent (Invitrogen) according to the manufacturer's instructions. Transfected cells were cultured for an additional 24 hours. Transfection efficiency between 30% to 50% was typically obtained. In some experiments, MelFB and CD40-B cells were electroporated using a Nucleofection™ system (Amaxa Biosystems; Gaithersburg, Md.) according to the manufacturer's instructions.

The HLA-DRβ1*0701-binding peptide gp100₁₇₀₋₁₉₀ (Lapointe R, et al., 2001. J. Immunol. 167:4758-4764) and the HLA-DRβ1*0701 control binding peptide Igk₁₈₈₋₂₀₂ (Chicz et al., 1993. J. Exp. Med. 178:27-47) were synthesized at the Surgery Branch (NCI/NIH). Recombinant gp100 protein was prepared as described previously (Touloukian et al., 2000. J. Immunol. 164:3535-3542). Recombinant NY-ESO-1 protein (Zeng et al., J. Immunol. 165:1153-1159), another tumor antigen, was used as a negative control. Peptide- or protein-pulsing of CD40-B cells (1×10⁵) was carried out in B-cell culture medium for 16 hours in 96-well flat-bottom plates.

### T Cell Assays

Gp100-specific T cell clones were analyzed for their capacity to recognize target cells, such as gp100-transfected 293T cells, melanoma cell lines or CD40-B pulsed with synthetic peptides or recombinant proteins. Target cells (1×10⁵) were co-cultured with either a specific CD4⁺ T cell clone (2×10⁴) or a specific CD8⁺ T cell clone (133 10⁵) in 200 ml of complete medium, in 96-well flat-bottom plates. Supernatants were harvested after 20 hours of incubation, and human Interferon-gamma (IFN-γ) was assayed by ELISA using conjugated antibodies (Endogen; Woburn, Mass.).

In some experiments, chloroquine (CHL) (Sigma; St-Louis, Mo.) at a concentration of 100 mM was added for 4 hours on target cells. Cells were washed once and fixed with 0.5% of formaldehyde for 5 min. Cells were then washed 3 times and co-cultured with T cells in 200 ml of complete medium in 96-well flat-bottom plates.

### Western Blotting

Protein extracts were prepared from gp100-transfected 293T cells at 4° C., for 20 min, in lysis buffer (20 mM Tris-HCl pH 8, 137 mM NaCl, 10% glycerol, 1% Triton X-100, 1 mM Na₃VO₄ and 2 mM EDTA) containing protease inhibitors (1 mM PMSF, 2 mM pepstatin A, 2 mM leupeptin) (all from Sigma). Cell debris were sedimented and discarded, and protein concentration was measured using a DC Protein Assay kit (Bio-Rad; Hercules, Calif.). Proteins were prepared and loaded (7.5 μg/well) on 10% SDS-polyacrylamide gel in a Mini-PROTEAN™ 3 system (Bio-Rad) according to the manufacturer's instructions. Proteins were transferred to Hybond™ ECL membranes (Amersham Pharmacia Biotech; Buckinghamshire, UK) and revealed by incubation with a goat gp100-specific antibody (dilution 1:200) (clone K-18; Santa Cruz; Santa Cruz, Calif.) or a mouse actin-specific antibody (dilution 1:4,000) (Chemicon; Temecula, Calif.), for 1 hour. Membranes were washed and re-incubated for 1 hour with secondary peroxidase-conjugated antibodies, chicken anti-goat (dilution 1:10,000) or goat anti-mouse (dilution 1:40,000) (both from Chemicon), before detection with ECL PIUS™ Western Blotting (Amersham Pharmacia Biotech).

### Confocal Microscopy

Cells were plated at 3×10⁵ cells/well on Poly-D-Lysine-treated coverslips (Sigma) in 12-well plates the day before transfection (when applicable) and cultured for an additional 24 hours. Before intracellular staining, cells were washed once with PBS (Invitrogen and Wisent) containing 0.5% BSA (Sigma), fixed and permeabilized with BD Cytofix/Cytoperm™ (BD Biosciences; Mississauga, ON) directly on coverslips for 20 minutes and washed twice with BD Perm/Wash™ Solution (BD Biosciences).

Permeabilized cells were stained with a gp100 -specific antibody (clone NK-1; Bio-Design; Saco, Me.), a LAMP-1-specific antibody (anti-CD107a; BD Biosciences) or a pan-MHC Class II (HLA-DR, P, Q)-specific antibody (clone TÜ39; BD Biosciences). After 30 minutes of incubation with the first antibody, cells were washed and re-incubated for 30 minutes with isotype-specific secondary antibodies conjugated with Alexa Fluor™-488 (green), −568 (red) or −647 (blue) (all from Molecular Probes; Eugene, Oreg.). Cells were then washed and the coverslips were mounted on microscope slides using Geltol (Immunon; Pittsburgh, Pa.). After overnight incubation at 4° C., the coverslips were sealed with nail polish.

Cells were observed under a Leica TCS-SP1™ confocal microscope (Leica Microsystems; Mannheim, Germany) fitted with an ×100 oil immersion objective, analyzed by Leica Confocal Software™ (LCS) and processed using Adobe Photoshop™ 7.0 (Adobe Systems Inc.; San Jose, Calif.).

### Flow Cytometry

293T cells were co-transfected by plasmids coding for GFP and gp100 mutants. Preliminary experiments in transient transfection confirmed that the same cells were co-transfected with 2 different plasmids, gp100 and GFP, for instance (FIG. 13). Twenty-four hours after transfection, cells were harvested with trypsin, distributed at >1×10⁵ cells/tube in 5 ml polystyrene round-bottom tubes and washed with PBS supplemented with 0.5% BSA. For intracellular staining, the cells were fixed and permeabilized with BD Cytofix/Cytoperm™ for 20 min, then washed twice with BD Perm/Wash™ Solution (both from BD Biosciences).

Intracellular and cell surface staining were performed using a gp100-specific antibody (clone NK-1) or an isotype-matched control (IgG2b; BD Biosciences). After 30 minutes of incubation, the cells were washed and re-incubated for 30 minutes with a phycoerythrin(PE)-conjugated isotype-specific secondary antibody (anti-mouse-R-PE; Molecular Probes). Cells were finally analyzed using a BD FACSCalibur™ flow cytometer (Becton Dickinson; Mississauga, ON). Only GFP-positive cells, which were also positive for gp100, were analyzed using the WinMDI™ 2.8 software. Gp100 cell surface expression was compared with total expression in permeabilized cells.

### Antigen-Specific Expansion of Autologous T Lymphocytes

Gp-M1 and gp-DKK1 plasmids were electroporated in CD40-activated B lymphocytes, which are APCs efficient in T cell stimulation (Lapointe et al., 2003. Cancer Res. 63: 2836-2843; Lapointe et al., 2004. Cancer Res. 64: 4056-4057; Schultze et al., 1997. J. Clin. Invest. 100: 2757-2765). Modified APCs were co-cultured with autologous, purified T lymphocytes to allow expansion of antigen-specific T cells. Individual cultures were re-stimulated 7-10 days later with antigen-expressing APCs, and interleukin (IL)-2 was added every 2-3 days thereafter. The specificity of individual cultures was evaluated by co-culture with autologous APC expressing the relevant construct or a control construct. Recognition by cultured T lymphocytes was monitored by interferon (IFN)-γ secretion evaluated by ELISA.

### Example 2

Exogenous and Endogenous gp100 can be Presented by MHC Class II Molecules

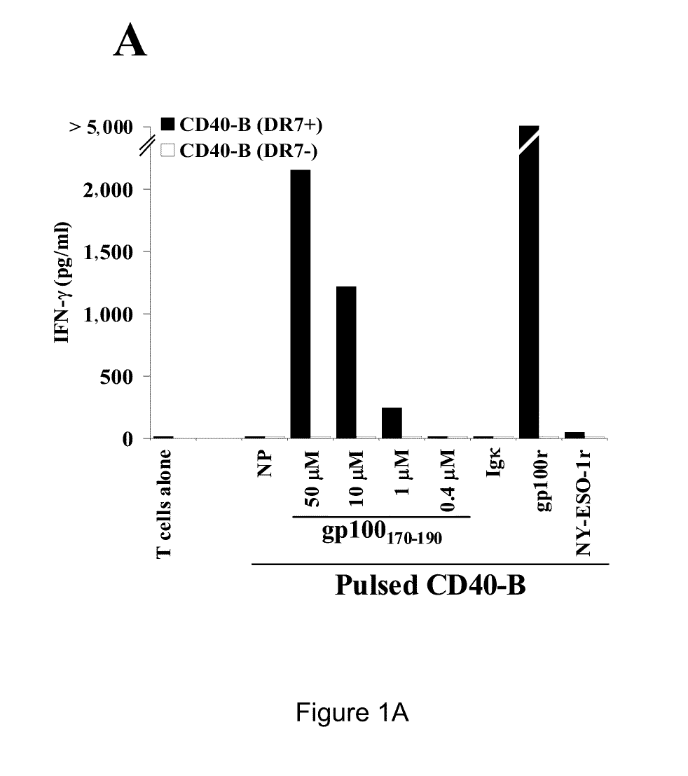

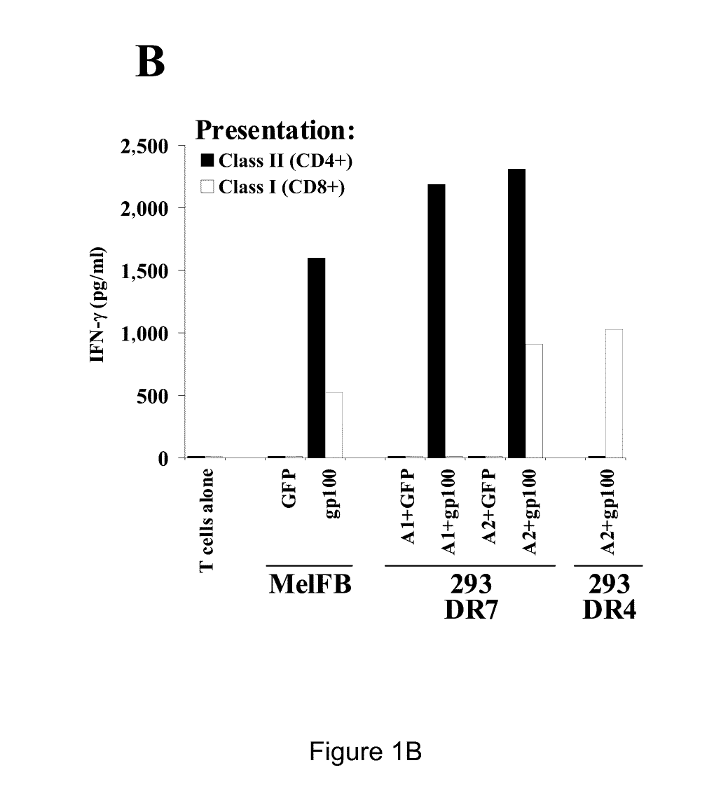

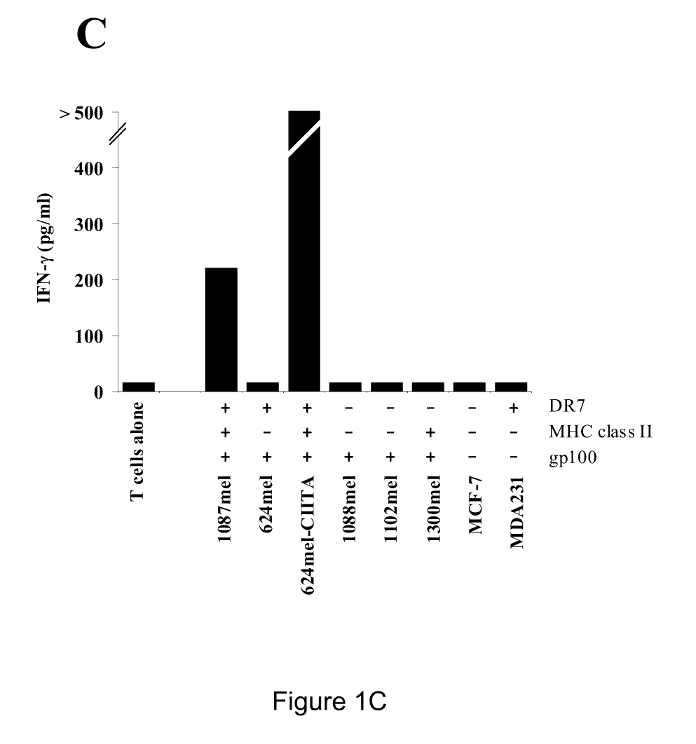

HLA-DRβ1*0701⁺ APCs pulsed with recombinant gp100, but not the DRβ1*0701 APCs alone, are recognized by the gp100-specific CD4⁺ T cell clone (Lapointe R, et al., 2001. J. Immunol. 167:4758-4764) (FIG. 1A). APCs pulsed with different amounts of gp100 peptide, corresponding to the DRβ1*0701 epitope (gp100₁₇₀₋₁₉₀), were also recognized in a dose-dependent manner, but DRβ1*0701+APC pulsed with either a different recombinant protein or a known DRβ1*0701-binding peptide derived from the immunoglobulin k light chain (Igk₁₈₈₋₂₀₂) were not recognized by the gp100-specific CD4⁺ T cell clone.

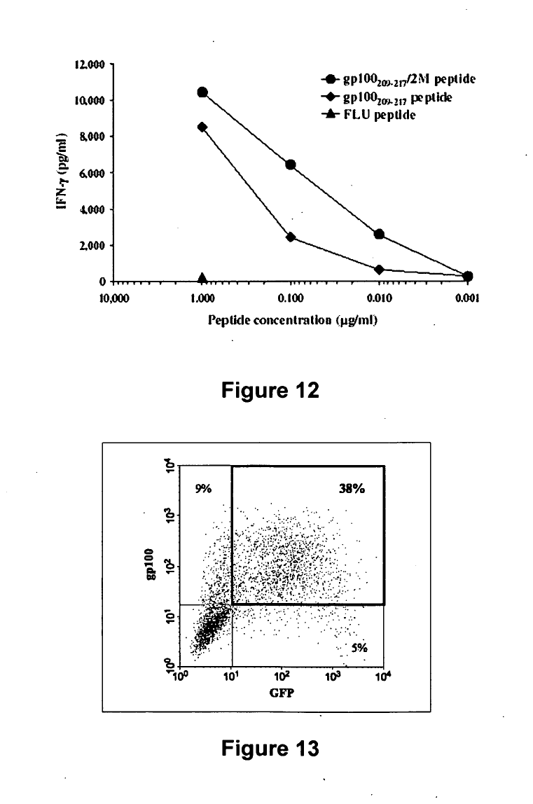

This gp100-specific CD4⁺ T cell clone was used to evaluate MHC class II-mediated presentation from endogenously-expressed gp100. HLA-DRβ1*0701+melanoma cells (MelFB), immuno-selected for the absence of gp100, and 293T cells were engineered to express gp100 or GFP. Only cells expressing gp100 and HLA-DRβ1*0701 were recognized (FIG. 1B). 293T cells expressing a control gene (GFP) or a different MHC class II molecule (HLA-DRβ1*0401) failed to induce the secretion of IFN-γ by the CD4⁺ T cell clone. In all cases, gp100 expression and MHC class I presentation were controlled by co-transfection of an HLA-A*0201 expression plasmid, and recognition was monitored by a CD8⁺ T cell clone specific to an HLA-A*0201 gp100 epitope (gp100₂₀₉₋₂₁₇) (Dudley et al., J. Immunother. 22:288-298). The amount of IFN-γ secretion by gp100-specific CD4⁺ (Lapointe R, et al., 2001. J. Immunol. 167:4758-4764) or CD8⁺ T cell clones correlates with the density of the peptide loaded on APCs (FIG. 12).

Also, melanoma cells expressing gp100 and the class II transactivator (CIITA), up-regulating invariant chain (Ii), HLA-DM and -DR molecules, were recognized by the CD4⁺ T cell clone (624 mel-CIITA, FIG. 1C), but not wild-type CIITA-cells not expressing HLA-class II molecules (i.e. 624 mel). Melanoma cells naturally expressing DRβ1*0701 and gp100 (1087 mel) were also recognized by the CD4⁺ T cell clone. Other melanoma cells expressing other MHC class II molecules, but not DRβ1*0701 (1300 mel), were not recognized, demonstrating that the recognition of APCs by the CD4⁺ T cell clone is HLA-DRβ1*0701-restricted.

The requirement for intracellular antigen processing for MHC class II presentation of endogenously-expressed gp100 was evaluated. To do so, target cells were treated with CHL, which inhibits the processing of exogenous antigen and MHC class II presentation by neutralizing the pH of endosomes. As shown in FIG. 1D (left panel), CHL treatment resulted in inhibition of MHC class II presentation of (1) exogenous gp100 by HLA-DRβ1*0701⁺ APCs pulsed with recombinant gp100 and (2) endogenous gp100 expressed by a melanoma cell line or HLA-DRβ1*0701⁺ 293T cells, indicating that intracellular antigen processing is involved in MHC class II presentation of gp100. This inhibition was not caused by CHL toxicity, since similar treatments of tumor cell lines did not inhibit MHC class I presentation of endogenous gp100 (FIG. 1D, right panel).

These results demonstrate that gp100 can be presented by MHC class 11 molecules from either classical exogenous or endogenous pathways.

### Example 3

Gp100 Localizes to LAMP-1+Endosomal Vesicles

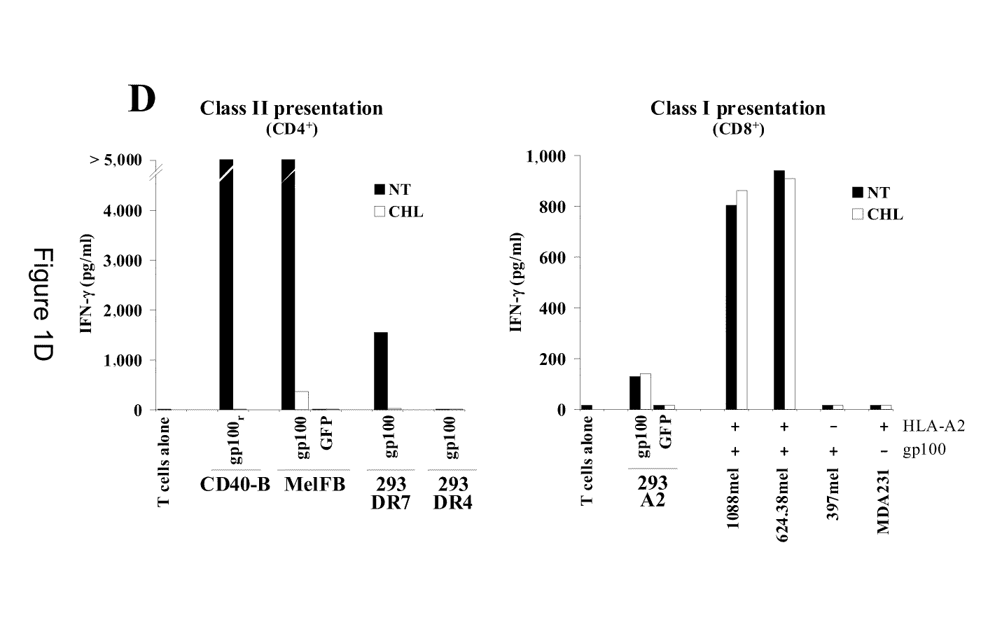

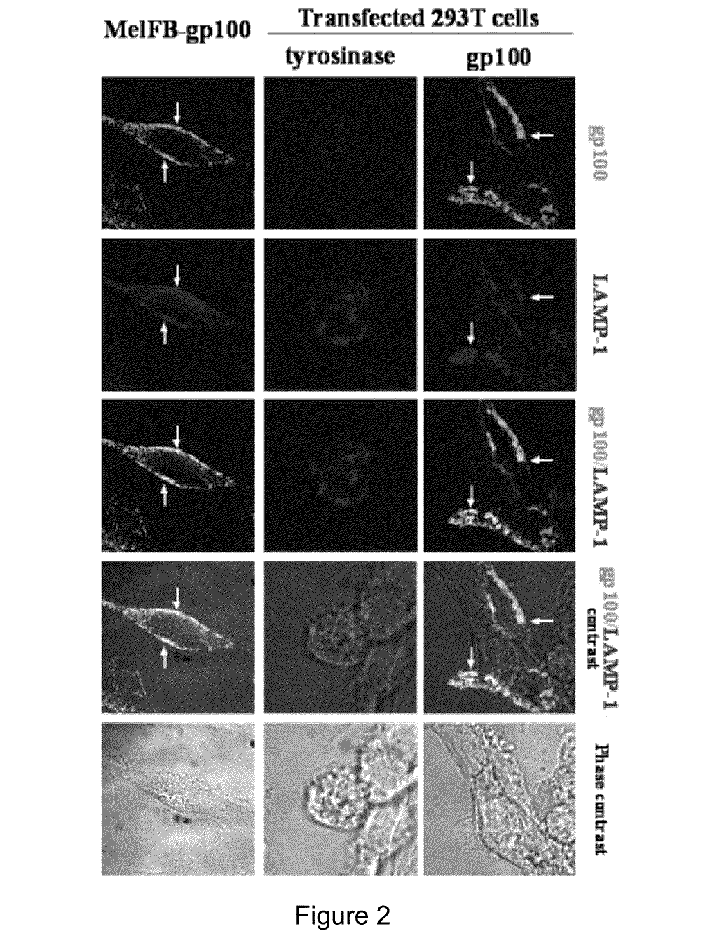

Gp100 localization experiments were carried out with laser scanning confocal microscopy. As shown in FIG. 2, gp100 appears to be localized in intracellular vesicles in both gp100-expressing melanoma cells and gp100-transfected 293T cells. Double staining was performed with anti-LAMP-1, a membrane glycoprotein enriched in the lysosomal membrane and found in endosomes/lysosomes and MIIC (Peters et al., 1991. Nature 349: 669-675; Calafat et al., 1994. J. Cell Biol. 126: 967-977). Double staining revealed that several vesicles staining positive for gp100 were also positive for LAMP-1, suggesting co-localization in endosomal compartments (white arrows). This experiment thus shows the trafficking of gp100 into endosomal compartments.

### Example 4

Mapping of Endosomal Targeting Sequences Involved in MHC Class II Presentation

To identify the region(s) of gp100 involved in MHC class II presentation, different deletion mutants of gp100 were generated. Plasmids encoding gp100 mutants and HLA-A*0201 were co-transfected in 293T cells expressing HLA-DRβ1*0701. MHC class I presentation was monitored with a CD8⁺ T cell clone specific to an HLA-A*0201 epitope from gp100 (gp100₂₀₉₋₂₁₇). MHC class II presentation was evaluated with a CD4⁺ T cell clone specific to an HLA-DRβ1*0701 epitope from gp100 (gp100₁₇₀₋₁₉₀). The different versions of deleted gp100 are illustrated in FIG. 3A (left panel). The mutations were designed to avoid the deletion of both MHC class I and class II epitopes; only the last 67 residues were modified, which is more than 350 amino acids downstream of the epitopes. As measured by IFN-γ secretion, gp100- and HLA-A*0201-transfected 293-DRβ1*0701⁺ cells (293 DR7/A2) were recognized by both CD4⁺ and CD8⁺ T cell clones, whereas gp100 and HLA-A*0101-transfected 293-DRβ1*0701⁺ cells (293 DR7/A1) failed to be recognized by the CD8⁺ T cell clone. Also, gp100 and HLA-A*0201-transfected 293-DRβ1*0401⁺ cells (293 DR4/A2) failed to be recognized by the CD4⁺ T cell clone. Although MHC class I-mediated presentation was similar for all gp100 mutants (FIG. 3A), deletions in the carboxy-terminus resulted in decreased MHC class II-restricted presentation. More specifically, deletion of the last 67 residues (gp100-NoTM) or internal deletion of the transmembrane domain (gp100-ΔTM) leads to a significant decrease in MHC class II presentation. The levels of IFN-γ secreted by the CD4⁺ T cell clone following stimulation with APCs transfected with the gp100 deletion mutants are then less than 10% as compared to the levels induced by the APCs transfected with wild-type gp100. However, as demonstrated by the MHC class II/class I presentation ratio (FIG. 3B), MHC class II presentation was not significantly affected by substitution of the transmembrane domain of gp100 by the transmembrane domain of CD8 (gp100-CD8). Deletion of the C-terminal portion of gp100 (from residue 650 to the end) (gp100-ΔLL), which comprises a putative di-leucine motif, minimally diminished MHC class II presentation, as illustrated by the MHC class II/class I presentation ratio (FIG. 3B). Further deletion in the carboxy-terminal sequence downstream of the transmembrane domain (gp100-TM) resulted in 45% IFN-γ secretion by the CD4⁺ T cell clone when compared to the full-length sequence (FIG. 3A).

Deletion of a sequence of 12 residues, including a tyrosine and 3 consecutive arginine residues, located immediately after the transmembrane domain (gp100-ΔYV), had minimal effect on MHC class II presentation (FIG. 3A). However, this deletion, combined with CD8-transmembrane substitution, (gp100-ΔYVCD8) abrogated MHC class II presentation, as illustrated by the MHC class II/class I presentation ratio (FIG. 3B). Finally, deletion of the first 20 amino-terminal residues (gp100-ΔSS) resulted in marked decrease MHC class II presentation with no significant changes in MHC class I presentation as compared to wild-type gp100 (FIG. 3A).

The observation that MHC class I presentation is equivalent for all gp100 mutants indicates that these mutants are expressed at similar levels in the cells, and thus that the differences in MHC class II presentation between some of the mutants and wild-type gp100 is caused by the mutation and not by differences in expression (FIG. 3A). A comparable expression level was further confirmed by analysis of gp100 expression by Western blotting (FIG. 3D). The expression level of gp100-ΔSS could not be evaluated by Western blotting, since the epitope recognized by the antibody is located in the amino-terminus. Clearly, the data demonstrated that a region located in the last 67 C-terminal residues of gp100, and more particularly a region within and/or in the vicinity of the putative transmembrane domain, as well as a portion comprised within the first 20 amino-terminal residues, are involved in gp100 MHC class II-mediated presentation.

### Example 5

Gp100 Cell Surface Expression Correlates with MHC Class II Presentation

Gp100 can possibly reach relevant endosomal compartments by 2 pathways for processing and loading into MHC class II molecules: 1) directly from the Golgi, and 2) by transiting to the cell surface following by internalization. The decrease in MHC class II presentation in gp100 mutants was not caused by increased endoplasmic reticulum (ER)/Golgi retention, since endoglycosidase H (EndoH) sensitivity patterns were similar for all gp100 mutants. Thus, to address the possibility of transition to the cell surface, gp100 cell surface expression was determined by flow cytometry and compared with total gp100 expression in permeabilized cells (FIG. 3E). All gated transfected cells were gp100+, and surface expression was detected in 59% of these cells. The results of gp100 cell surface expression for all mutants is summarized in FIG. 3B. Cells transfected with a plasmid encoding gp100-ΔLL express gp100 at their cell surface (FIG. 3E). In contrast, cells transfected with gp100-NoTM, gp100-ATM, gp100-ΔSS or gp100-ΔYVCD8 failed to mobilize gp100 to the cell surface. Consequently, as illustrated in FIG. 3B, there was a direct correlation between gp100 cell surface expression and the MHC class II/class I presentation ratio.

Gp100 cell surface and total expression was assessed in 8 different melanoma cell lines. Gp100 expression was detected in 6 of 7 melanoma cell lines tested (excluding MelFB) (FIG. 4), and gp100 cell surface expression was observed in 3 of these 6 melanoma cell lines. Gp100 cell surface expression was also noted in a gp100⁻ melanoma cell line (MelFB) engineered to express gp100.

### Example 6

Endosomal Localization of gp100 Mutants Presented by MHC Class II

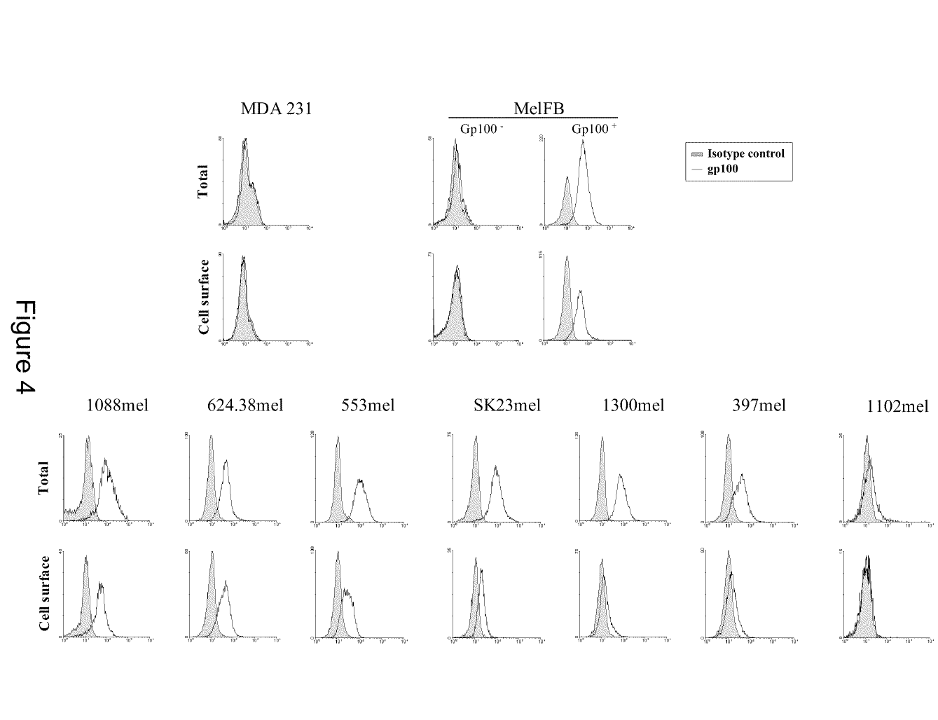

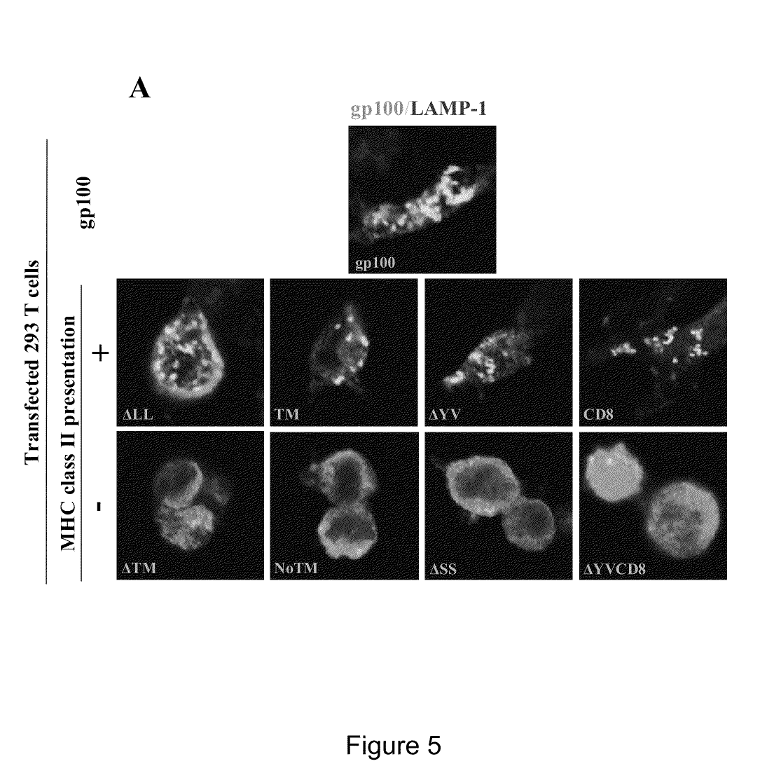

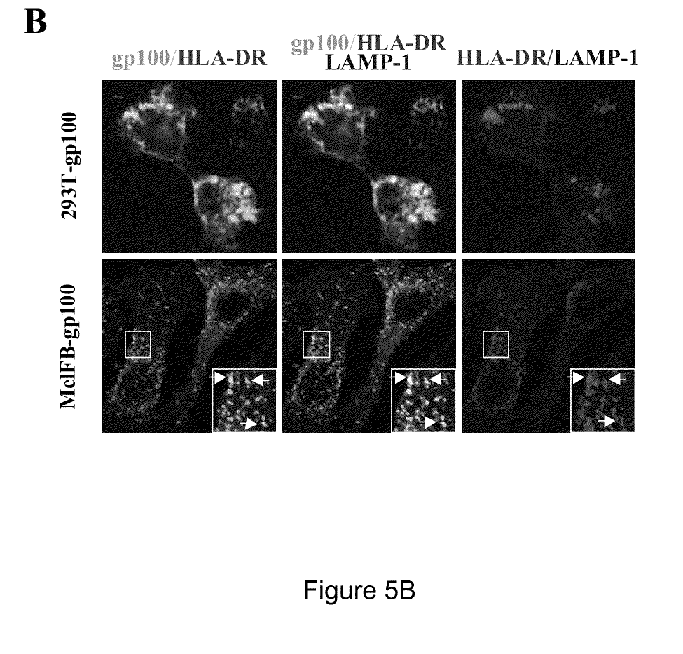

The different gp100 mutants were further characterized by gp100 localization experiments with laser scanning confocal microscopy of transfected 293T cells stained for gp100 and LAMP-1. Gp100 mutants which showed proper MHC class II presentation (FIG. 3) were located in intracellular vesicles, as shown by co-localization with LAMP-1, similar to wild-type gp100 (FIG. 5A). In contrast, the gp100 mutants which had decreased MHC class II presentation, showed no specific vesicular localization, and no co-localization with LAMP-1, demonstrating that region(s) located within the deleted sequences are involved in gp100 trafficking. Gp100-ΔLL-transfected cells show higher gp100 cell surface expression as compared to wild-type gp100-transfected cells, confirming the results obtained by flow cytometry (FIG. 3E).

To further confirm that LAMP-1⁺ endosomes co-localizing with gp100 are MHC class II compartments (MIIC), gp100-transfected 293T cells and melanoma cells were stained using an anti-gp100, an anti-LAMP-1 and an anti-HLA-DR, and laser scanning confocal microscopy was performed. As shown in FIG. 5B, LAMP-1⁺ vesicles containing gp100 (left image) were also positive for HLA-DR (central image), indicating that they are MIIC. LAMP-1⁺/HLA-DR⁻ vesicles in MelFB may represent melanosomes.

### Example 7

Sequences from gp100 Mobilize GFP to Endosomes and Allow the Presentation of Minimal Class II and Class I Epitopes

Sequences from gp100 were cloned in fusion with GFP, transfected in 293T cells engineered to express MHC class II and accessory molecules, and laser scanning confocal microscopy was performed. As presented in FIG. 6A, wild-type GFP showed no particular mobilization. However, GFP in fusion with the first 20 N-terminal and the last 67 C-terminal amino acids from gp100 (gp100/GFP) co-localized with LAMP-1 (white arrows).

To link endosomal localization to MHC class II-mediated presentation, a short sequence from gp100, corresponding to minimal class II and class I epitopes, was inserted after GFP in the gp100/GFP construct described above. Plasmids encoding this chimeric protein (gp/GFP+epit) and HLA-A*0201 or A*0101 were co-transfected in 293T cells expressing HLA-DRβ1*0701 or DRβ1*0401. GFP expression was confirmed by flow cytometry, and vesicular mobilization was studied by fluorescence microscopy. As presented in FIG. 6B, 293-DRβ1*0701 cells transfected by plasmids encoding gp/GFP+epit or wild-type gp100 were recognized by the CD4⁺ T cell clone. 293-DRβ1*0401 failed to stimulate the CD4⁺ T cell clone. 293-DRβ1*0201 cells transfected by plasmids encoding gp/GFP+epit and full-length gp100 were efficiently recognized by the CD8⁺ T cell clone, whereas the negative control, HLA-A*0101-transfected 293T cells, failed to be recognized. Presentation of gp100-MHC class II epitope was further confirmed in melanoma (MelFB; FIG. 6C) and APCs (CD40-B).

These experiments confirm the role of regions located in the N-terminal and C-terminal portions of gp100 in: 1) mobilization to endosomes, and 2) MHC class II-mediated presentation.

### Example 8

Expansion of Antigen-Specific T Lymphocytes by Cells Transfected with Antigens Fused with Regions from gp100.

Chimeric proteins were generated in expression plasmids with 2 antigens: a candidate tumour antigen (Dickkopf-1 or DKK1) (Forget et al., 2007. Br. J. Cancer 96: 646-653), and a viral antigen (M1 matrix protein from influenza) (Leclerc et al., 2007. J. Virol. 81: 1319-1326), each cloned in fusion with portions of gp100 (residues 1-20 and 578-661 of gp100, FIG. 8) in a CMV promoter-based plasmid (pCDNA3 from Invitrogen).

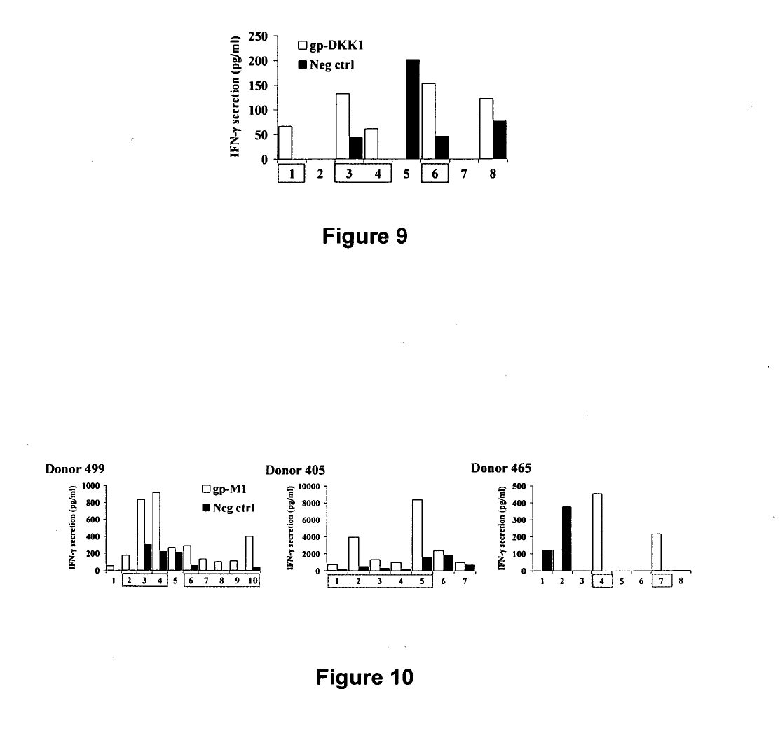

T lymphocytes specific for DKK1 were expanded in 4 wells on 8 individual cultures using a gp-DKK1 construct transfected in autologous APC from normal donor #499 (FIG. 9). T lymphocytes specific for influenza M1 were also expanded in cultures from three different donors (donors #499, 405 and 465) using a gp-M1 construct transfected in autologous APC (FIG. 10).

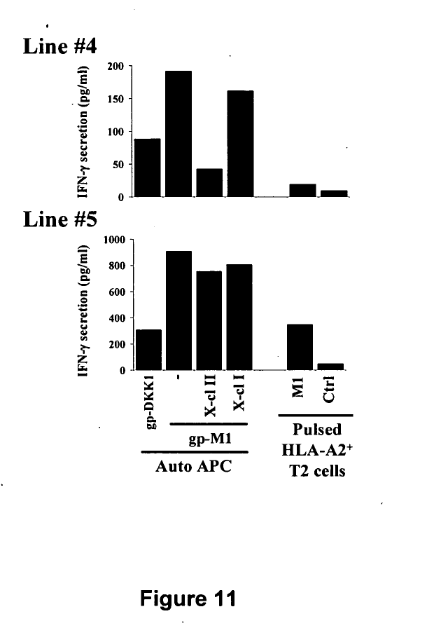

To determine if both CD4⁺ and CD8⁺ T cell lines were expanded, confirming antigenic presentation by MHC class II and class I molecules respectively, antibodies blocking presentation by MHC class I and MHC class II were added to target cells 20 minutes before the addition of cultured T cell lines. As shown in FIG. 11, recognition of APC expressing gp-M1 by line #4 from donor #405 was significantly abrogated in the presence of an anti-MHC class II antibody, demonstrating that this specific T cell line recognized an epitope presented by MHC class II. Conversely, line #5 was weakly blocked by either antibody, suggesting that this is a mixed CD4/CD8 T cell population. Since donor #405 was HLA-A2⁺, both lines were also co-cultured with T2 cells pulsed with a known MHC class I/HLA-A2 epitope from M1 (GILGFVFTL; SEQ ID NO:9). Line #5 was reactive against this HLA-A2 epitope, confirming the presence of T cells reacting against this MHC class I epitope.

Although the present invention has been described hereinabove by way of specific embodiments thereof, it can be modified, without departing from the spirit and nature of the subject invention as defined in the appended claims.

1. A chimeric nucleic acid comprising:
(a) a first domain comprising a nucleic acid encoding a signal peptide; and
(b) a second domain comprising a nucleic acid encoding a polypeptide comprising (i) a transmembrane domain and (ii) an antigen or an epitope thereof,
wherein at least one of the signal peptide and the transmembrane domain is that of gp100, and wherein said antigen or epitope is heterologous to at least one of said signal peptide and said transmembrane domain.

2. The chimeric nucleic acid of claim 1, wherein said signal peptide comprises the sequence of SEQ ID NO:3.

3. The chimeric nucleic acid of claim 2, wherein said nucleic acid encoding a signal peptide comprises the sequence of SEQ ID NO:12.

4. The chimeric nucleic acid of claim 1, wherein said polypeptide comprises the transmembrane domain of gp100 or of CD8.

5. The chimeric nucleic acid of claim 3, wherein said transmembrane domain comprises the sequence of SEQ ID NO:1 or 4.

6. The chimeric nucleic acid of claim 5, wherein said nucleic acid encoding a transmembrane domain comprises the sequence of SEQ ID NO:10 or 13.

7. The chimeric nucleic acid of claim 1, wherein said polypeptide comprises the sequence of SEQ ID NO:2.

8. The chimeric nucleic acid of claim 7, wherein said nucleic acid encoding said polypeptide comprises the sequence of SEQ ID NO:11.

9. The chimeric nucleic acid of claim 4, wherein said transmembrane domain is that of CD8 and further comprises at its C-terminal end the sequence of SEQ ID NO:5.

10. The chimeric nucleic acid of claim 9, wherein said nucleic acid encoding said polypeptide further comprises the sequence of SEQ ID NO:14.

11. The chimeric nucleic acid of claim 1, wherein said antigen or epitope thereof is derived from a tumour antigen.

12. The chimeric nucleic acid of claim 1, wherein said antigen or epitope thereof is derived from a viral antigen.

13. A polypeptide encoded by the chimeric nucleic acid of claim 1.

14. A vector comprising the nucleic acid of claim 1.

15. A cell comprising the nucleic acid of claim 1 or the vector of claim 14.

16. A composition comprising the chimeric nucleic acid of claim 1 or the polypeptide of claim 13, and a pharmaceutically acceptable carrier or excipient.

17. The composition of claim 16, further comprising an adjuvant.

18. A method for inducing or enhancing an immune response against an antigen in a subject, comprising administering to said subject a composition comprising a chimeric nucleic acid and an adjuvant, said chimeric nucleic acid comprising:
(a) a first domain comprising a nucleic acid encoding a signal peptide; and
(b) a second domain comprising a nucleic acid encoding a polypeptide comprising (i) a transmembrane domain and (ii) said antigen or an epitope thereof, wherein said antigen or epitope is heterologous to at least one of said signal peptide and said transmembrane domain.

19. A method for enhancing MHC class-II presentation of an antigenic epitope in a cell, comprising transfecting or transforming said cell with a chimeric nucleic acid comprising:
(a) a first domain comprising a nucleic acid encoding a signal peptide; and
(b) a second domain comprising a nucleic acid encoding a polypeptide comprising (i) a transmembrane domain and (ii) said antigenic epitope, wherein said antigenic epitope is heterologous to at least one of said signal peptide and said transmembrane domain.

20. The method of claim 18, wherein said signal peptide is a signal peptide from gp100.

21. The method of claim 20, wherein said signal peptide comprises the sequence of SEQ ID NO:3.

22. The method of claim 21, wherein said nucleic acid encoding a signal peptide comprises the sequence of SEQ ID NO:12.

23. The method of claim 18, wherein said polypeptide comprises the transmembrane domain of gp100 or of CD8.

24. The method of claim 23, wherein said transmembrane domain comprises the sequence of SEQ ID NO:1 or SEQ ID NO:4.

25. The chimeric nucleic acid of claim 24, wherein said nucleic acid encoding a transmembrane domain comprises the sequence of SEQ ID NO:10 or 13.

26. The method of claim 18, wherein said antigen is a tumour antigen.

27. The method of claim 26, wherein the subject has a tumour expressing the tumour antigen.

28. The method of claim 18, wherein said antigen or epitope thereof is derived from a viral antigen.

29. The method of claim 28, wherein the subject is susceptible to or has a viral infection expressing the viral antigen.

30. The method of claim 19, wherein said cell is an antigen-presenting cell (APC).

31. The method of claim 30, wherein said APC is a dendritic cell.

32. A method for inducing or enhancing an immune response against an antigen in a subject, comprising administering to said subject cells transfected or transformed with the chimeric nucleic acid of claim 1.

33. A method for inducing or enhancing an immune response against an antigen in a subject, comprising administering to said subject T lymphocytes that have been co-cultured with cells transfected or transformed with the chimeric nucleic acid of claim 1.

34. A method of expanding T lymphocytes comprising co-culturing the lymphocytes with cells transfected or transformed with the chimeric nucleic acid of claim 1.

35. The method of claim 32, wherein said cells are autologous to the subject.

36. The method of claim 33, wherein said T lymphocytes and said cells are autologous to the subject.

37. The method of claim 34, wherein said T lymphocytes and said cells are autologous to the subject.


SEQUENCE LISTING (20 sequences)

SEQ ID NO: 1 - protein, 21 aa, Homo sapiens
QVPLIVGILLVLMAVVLASLI

SEQ ID NO: 2 - protein, 67 aa, Homo sapiens
QVPLIVGILLVLMAVVLASLIYRRRLMKQDFSVPQLPHSSSHWLRLPRIFCSCPIGENSPLLSGQQV

SEQ ID NO: 3 - protein, 20 aa, Homo sapiens
MDLVLKRCLLHLAVIGALLA

SEQ ID NO: 4 - protein, 22 aa, Homo sapiens
IYIWAPLAGTCGVLLLSLVITL

SEQ ID NO: 5 - protein, 12 aa, Homo sapiens
YRRRLMKQDFSV

SEQ ID NO: 6 - protein, 84 aa, Homo sapiens
AVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 7 - DNA, 2130 bp, Homo sapiens
  CDS (22)..(2004)
cgcggaatccggaagaacacaatggatctggtgctaaaaagatgccttcttcatttggctgtgataggtgctttgctggctgtgggggctacaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggccaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactgaccaggtgcctttctccgtgagcgtgtcccagttgcgggccttggatggagggaacaagcacttcctgagaaatcagcctctgacctttgccctccagctccatgaccccagtggctatctggctgaagctgacctctcctacacctgggactttggagacagtagtggaaccctgatctctcgggcacttgtggtcactcatacttacctggagcctggcccagtcactgcccaggtggtcctgcaggctgccattcctctcacctcctgtggctcctccccagttccaggcaccacagatgggcacaggccaactgcagaggcccctaacaccacagctggccaagtgcctactacagaagttgtgggtactacacctggtcaggcgccaactgcagagccctctggaaccacatctgtgcaggtgccaaccactgaagtcataagcactgcacctgtgcagatgccaactgcagagagcacaggtatgacacctgagaaggtgccagtttcagaggtcatgggtaccacactggcagagatgtcaactccagaggctacaggtatgacacctgcagaggtatcaattgtggtgctttctggaaccacagctgcacaggtaacaactacagagtgggtggagaccacagctagagagctacctatccctgagcctgaaggtccagatgccagctcaatcatgtctacggaaagtattacaggttccctgggccccctgctggatggtacagccaccttaaggctggtgaagagacaagtccccctggattgtgttctgtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtcaagaagcaggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaatagccccctcctcagtgggcagcaggtctgagtactctcatatgatgctgtgattttcctggagttgacagaaacacctatatttcccccagtcttccctgggagactactattaactgaaataaatactcagagcctgaaaaaaaaaaaaaaa

SEQ ID NO: 8 - protein, 661 aa, Homo sapiens
MDLVLKRCLLHLAVIGALLAVGATKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRALVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIMPGQEAGLGQVPLIVGILLVLMAVVLASLIYRRRLMKQDFSVPQLPHSSSHWLRLPRIFCSCPIGENSPLLSGQQV

SEQ ID NO: 9 - protein, 9 aa, Influenza virus
GILGFVFTL

SEQ ID NO: 10 - DNA, 63 bp, Homo sapiens
caggttccgctgatcgtgggcatcttgctggtgttgatggctgtggtccttgcatctctgata

SEQ ID NO: 11 - DNA, 201 bp, Homo sapiens
caggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaatagccccctcctcagtgggcagcaggtc

SEQ ID NO: 12 - DNA, 60 bp, Homo sapiens
atggatctggtgctaaaaagatgccttcttcatttggctgtgataggtgctttgctggct

SEQ ID NO: 13 - DNA, 66 bp, Homo sapiens
atctacatctgggcgcccttggccgggacttgtggggtccttctcctgtcactggttatcaccctt

SEQ ID NO: 14 - DNA, 36 bp, Homo sapiens
tataggcgcagacttatgaagcaagacttctccgta

SEQ ID NO: 15 - DNA, 252 bp, Homo sapiens
gcagtggtcagcacccagcttatcatgcctggtcaagaagcaggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaatagccccctcctcagtgggcagcaggtc

SEQ ID NO: 16 - protein, 6 aa, Homo sapiens
ENSPLL

SEQ ID NO: 17 - protein, 4 aa, Homo sapiens
AGLG

SEQ ID NO: 18 - protein, 15 aa, Homo sapiens
YRRRLMKQDFSVPQL

SEQ ID NO: 19 - protein, 38 aa, Homo sapiens
RGLDFACDIYIWAPLAGTCGVLLLSLVITLYCNHRNRR

SEQ ID NO: 20 - protein, 75 aa, Homo sapiens
PGQEAGLGQVPLIVGILLVLMAVVLASLIYRRRLMKQDFSVPQLPHSSSHWLRLPRIFCSCPIGENSPLLSGQQV